(19) United States
(12) Patent Application Publication
(10) Pub. No.: US 2025/0214983 A1
(43) Pub. Date: Jul. 3, 2025

(54) 1,3,4-OXADIAZOLE TRIAZOLE COMPOUNDS AS HISTONE DEACETYLASE 6 INHIBITOR, AND PHARMACEUTICAL COMPOSITION COMPRISING THE SAME

(71) Applicant: Chong Kun Dang Pharmaceutical Corp., Seoul (KR)

(72) Inventors: Jae Kwang Lee, Gyeonggi-do (KR); Jaeki Min, Gyeonggi-do (KR); Jinkyung In, Gyeonggi-do (KR); Yi Hyun Kim, Gyeonggi-do (KR); Bomi Jeon, Gyeonggi-do (KR); Hyunjin Michael Kim, Gyeonggi-do (KR)

(21) Appl. No.: 18/881,499

(22) PCT Filed: Jul. 13, 2023
(86) PCT No.: PCT/IB2023/057181

§ 371 Date: Jan. 6, 2025

(30) Foreign Application Priority Data
Jul. 15, 2022 (KR) 10-2022-0087805

(51) Int. Cl. C07D 413/14; A61K 31/4439; A61K 31/444; A61K 31/4545; C07D 417/14

(52) U.S. Cl. CPC C07D 413/14; A61K 31/4439; A61K 31/444; A61K 31/4545; C07D 417/14

(57) ABSTRACT

The present invention relates to a novel compound having a histone deacetylase 6 (HDAC6) inhibitory activity, stereoisomers thereof, pharmaceutically acceptable salts thereof, a use thereof in the manufacture of a medicament, a pharmaceutical composition comprising the same, a preventive or therapeutic method thereof, and a method for preparing novel 1,3,4-oxadiazole triazol, wherein a novel compound having a selective HDAC6 inhibitory activity is represented by following formula I.

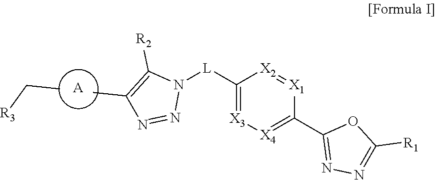

### TECHNICAL FIELD

The present invention relates to a novel compound having a histone deacetylase 6 (HDAC6) inhibitory activity, stereoisomers thereof, pharmaceutically acceptable salts thereof, a use thereof in the manufacture of a preventive or therapeutic medicament, a pharmaceutical composition comprising the same, a preventive or therapeutic method thereof, and a method for preparing the same.

### BACKGROUND ART

In cells, a post-translational modification such as acetylation serves as a very important regulatory module at the hub of biological processes, and is also strictly controlled by a number of enzymes. As a core protein constituting chromatin, histone functions as an axis, around which DNA winds, and thus helps a DNA condensation. In addition, a balance between acetylation and deacetylation of histone plays a very important role in gene expression.

As an enzyme for removing an acetyl group from lysine residue of histone protein, which constitutes chromatin, histone deacetylase (HDAC) is known to be associated with gene silencing and induce a cell cycle arrest, angiogenic inhibition, immunoregulation, apoptosis, etc. (Hassig et al., Curr. Opin. Chem. Biol. 1997, 1, 300-308). Moreover, it is reported that the inhibition of HDAC enzyme functions induces cancer cells into committing apoptosis for themselves by lowering an activity of cancer cell survival-related factors and activating cancer cell death-related factors in the body (Warrell et al., J. Natl. Cancer Inst. 1998, 90, 1621-1625).

For humans, 18 HDACs are known and classified into four classes according to homology with yeast HDAC. In this case, eleven HDACs using zinc as a cofactor may be divided into three groups: Class I (HDAC1, 2, 3, 8), Class II (IIa: HDAC4, 5, 7, 9; IIb: HDAC6, 10) and Class IV (HDAC11). Further, seven HDACs of Class III (SIRT 1-7) use NAD+ as a cofactor instead of zinc (Bolden et al., Nat. Rev. Drug Discov. 2006, 5 (9), 769-784).

Various HDAC inhibitors are now in a preclinical or clinical development stage, but only non-selective HDAC inhibitors have been known as an anti-cancer agent so far. Vorinostat (SAHA) and romidepsin (FK228) have obtained an approval as a therapeutic agent for cutaneous T-cell lymphoma, while panobinostat (LBH-589) has won an approval as a therapeutic agent for multiple myeloma. However, it is known that the non-selective HDAC inhibitors generally bring about side effects such as fatigue, nausea and the like at high doses (Piekarz et al., Pharmaceuticals 2010, 3, 2751-2767). It is reported that the side effects are caused by the inhibition of class I HDACs. Due to the side effects, etc., the non-selective HDAC inhibitors have been subject to restriction on drug development in other fields than an anticancer agent (Witt et al., Cancer Letters 277, (2009), 8-21).

Meanwhile, it is reported that the selective inhibition of class II HDACs would not show toxicity, which have occurred in the inhibition of class I HDACs. In case of developing the selective HDAC inhibitors, it would be likely to solve side effects such as toxicity, etc., caused by the non-selective inhibition of HDACs. Accordingly, there is a chance that the selective HDAC inhibitors may be developed as an effective therapeutic agent for various diseases (Matthias et al., Mol. Cell. Biol. 2008, 28, 1688-1701).

HDAC6, one of the class IIb HDACs, is known to be mainly present in cytoplasma and be involved in the deacetylation of a number of non-histone substrates (HSP90, cortactin, etc.) including a tublin protein (Yao et al., Mol. Cell 2005, 18, 601-607). HDAC6 has two catalytic domains, in which a zinc finger domain of C-terminal may bind to an ubiquitinated protein. HDAC6 is known to have a number of non-histone proteins as a substrate, and thus play an important role in various diseases such as cancer, inflammatory disease, autoimmune disease, neurological disease, neurodegenerative disorder and the like (Santo et al., Blood 2012 119, 2579-2589; Vishwakarma et al., International Immunopharmacology 2013, 16, 72-78; Hu et al., J. Neurol. Sci. 2011, 304, 1-8).

A structural feature that various HDAC inhibitors have in common is comprised of a cap group, a linker and a zinc binding group (ZBG) as shown in a following structure of vorinostat. Many researchers have conducted a study on the inhibitory activity and selectivity with regard to enzymes through a structural modification of the cap group and the linker. Out of the groups, it is known that the zinc binding group plays a more important role in the enzyme inhibitory activity and selectivity (Wiest et al., J. Org. Chem. 2013 78:5051-5055; Methot et al., Bioorg. Med. Chem. Lett. 2008, 18, 973-978).

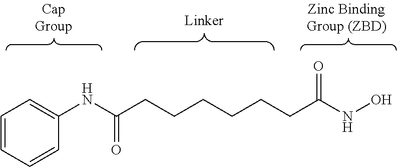 [SMILES: O=C(CCCCCCC(=O)Nc1ccccc1)NO]

Most of said zinc binding group is comprised of hydroxamic acid or benzamide, out of which hydroxamic acid derivatives show a strong HDAC inhibitory effect, but have a problem with low bioavailability and serious off-target activity. Benzamide contains aniline, and thus has a problem in that it may produce toxic metabolites in vivo (Woster et al., Med. Chem. Commun. 2015, online publication).

Accordingly, unlike the non-selective inhibitors having side effects, there is a need to develop a selective HDAC6 inhibitor, which has a zinc binding group with improved bioavailability, while causing no side effects in order to treat cancer, inflammatory disease, autoimmune disease, neurological disease, neurodegenerative disorder and the like.

### RELATED ART REFERENCES

### Patent Documents

International Patent Publication No. WO 2011/091213 (publicized on Jul. 28, 2011): ACY-1215
International Patent Publication No. WO 2011/011186 (publicized on Jan. 27, 2011): Tubastatin
International Patent Publication No. WO 2013/052110 (publicized on Apr. 11, 2013): Sloan-K
International Patent Publication No. WO 2013/041407 (publicized on Mar. 28, 2013): Cellzome
International Patent Publication No. WO 2013/134467 (publicized on Sep. 12, 2013): Kozi
International Patent Publication No. WO 2013/008162 (publicized on Jan. 17, 2013): Novartis
International Patent Publication No. WO 2013/080120 (publicized on Jun. 6, 2013): Novartis
International Patent Publication No. WO 2013/066835 (publicized on May 10, 2013): Tempero
International Patent Publication No. WO 2013/066838 (publicized on May 10, 2013): Tempero
International Patent Publication No. WO 2013/066833 (publicized on May 10, 2013): Tempero
International Patent Publication No. WO 2013/066839 (publicized on May 10, 2013): Tempero

### DISCLOSURE

### Technical Problem

An object of the present invention is to provide a compound having a selective HDAC6 inhibitory activity, stereoisomers thereof or pharmaceutically acceptable salts thereof.

Another object of the present invention is to provide a pharmaceutical composition including a compound having a selective HDAC6 inhibitory activity, stereoisomers thereof or pharmaceutically acceptable salts thereof.

Still another object of the present invention is to provide a method for preparing the same.

Still another object of the present invention is to provide a pharmaceutical composition for preventing or treating HDAC6-mediated diseases.

Still another object of the present invention is to provide a use thereof in the manufacture of a medicament for preventing or treating HDAC6-mediated diseases.

Still another object of the present invention is to provide a method for preventing or treating HDAC6-mediated diseases, including administering a therapeutically effective amount of the compound, stereoisomers thereof or pharmaceutically acceptable salts thereof.

Still another object of the present invention is to provide a use thereof for preventing or treating HDAC6-mediated diseases.

### Technical Solution

The present inventors have found an oxadiazole compound having a histone deacetylase 6 (HDAC6) inhibitory activity and have used the same in preventing or treating HDAC6-mediated diseases, thereby completing the present invention.

Hereinafter, the present invention will be described in more detail. All the combinations of various elements disclosed in the present invention fall within the scope of the present invention. In addition, it cannot be seen that the scope of the present invention is limited to the specific description below.

### Compound Represented by Formula I

(1) The present invention provides a 1,3,4-oxadiazole triazole compound represented by formula I below, stereoisomers thereof or pharmaceutically acceptable salts thereof:

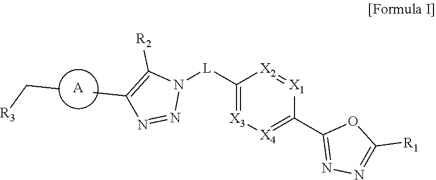

in above formula I,
X₁, X₂, X₃ and X₄ are each independently CH or N, in which at least one of X₁ to X₄ is N;
R₁ is CF₂H;
L is C1-C2 alkylene;
R₂ is H or C1-C5 alkyl;
A is C6-C12 aryl or 5 to 6 membered heteroaryl, in which at least one H of C6-C12 aryl is substituted with halogen;
R₃ is —NR₄R₅ or

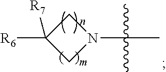

R₄ and R₅ are each independently H or C1-C6 alkyl;
R₆ and R₇ are each independently H, halogen, C1-C6 alkyl or C1-C6 haloalkyl; and
n and m are each independently 1 or 2.

In the present invention, halogen may be F, Cl, Br or I.

In the present invention, Cx-Cy (in which x and y are each an integer of 1 or greater) may represent a range of carbon atoms included in a corresponding substituent.

In the present invention, alkylene may refer to a divalent functional group derived from a linear or branched saturated hydrocarbon. For example, C1 alkylene may be methylene.

In the present invention, aryl may refer to a monocyclic aromatic or polycyclic aromatic functional group consisting of carbon and hydrogen only. For example, aryl may include phenyl, naphthyl, and the like.

In the present invention, heteroaryl may refer to a monocyclic or polycyclic heterocycle in which at least one carbon is substituted with a heteroatom, and examples of the heteroatom may include nitrogen (N), oxygen (O), sulfur(S), and the like. When heteroaryl includes at least two heteroatoms, the two heteroatoms or more may be the same or different from each other. For example, heteroaryl may include thiophenyl, pyridinyl, or thiazolyl.

In the present invention, haloalkyl may refer to a functional group in which at least one of H of alkyl, which is a monovalent functional group derived from a linear or branched saturated hydrocarbon, is substituted with halogen. For example, haloalkyl may include —CF₃, —CH₂—CF₃, —CHF—CH₃, —CF₂H, —CFH₂ and the like.

In the present invention, “ [symbol] ” may represent a connected part.
(2) In above (1), X₁, X₃ and X₄ of above formula I may be each CH, and X₂ may be N.
(3) In above (1) or (2), A of above formula I may be phenyl in which one hydrogen is substituted with halogen, or may be 5 to 6 membered heteroaryl including at least one heteroatom selected from N and S.
(4) In one of above (1) to (3), 5 to 6 membered heteroaryl may include thiophenyl, pyridinyl or thiazolyl.
(5) In one of above (1) to (4), there may be provided the 1,3,4-oxadiazol triazole compound according to the present invention, in which:
X₁, X₃ and X₄ of above formula I are each CH, and X₂ is N;
L is C1 alkylene; and
R₁, R₂, A and R₃ include the same compound as defined in above formula I, respectively.
(6) In above (1), there may be provided the 1,3,4-oxadiazole triazole compound according to the present invention, in which:
X₁ to X₄, R₁, L and R₂ of above formula I are the same as defined in above formula I;
A is C6 aryl, in which at least one H of C6 aryl is substituted with halogen;
R₃ is —NR₄R₅ or

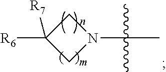

R₄ and R₅ are each independently C1-C6 alkyl;
R₆ and R₇ are each independently H or C1-C6 alkyl; and
n and m are each independently 1 or 2.
(7) In above (1) or (2), there may be provided the 1,3,4-oxadiazole triazole compound according to the present invention, in which:
X₁ to X₄, R₁, L and R₂ of above formula I are the same as defined in above formula I;
A is 6-membered heteroaryl;
R₃ is

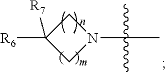

R₆ and R₇ are each independently H or C1-C6 alkyl; and
n and m are each independently 1 or 2.
(8) In above (1) or (2), there may be provided the 1,3,4-oxadiazole triazole compound according to the present invention, in which:
X₁ to X₄, R₁, L and R₂ of above formula I are the same as defined in above formula I;
A is 5-membered heteroaryl;
R₃ is —NR₄R₅ or

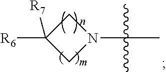

R₄ and R₅ are each independently C1-C6 alkyl;
R₆ and R₇ are each independently H, halogen, C1-C6 alkyl or C1-C6 haloalkyl; and
n and m are each independently 1 or 2.

In the present invention, pharmaceutically acceptable salts may refer to the salts conventionally used in a pharmaceutical industry, for example, inorganic ion salts prepared from calcium, potassium, sodium, magnesium or the like; inorganic acid salts prepared from hydrochloric acid, nitric acid, phosphoric acid, bromic acid, iodic acid, perchloric acid, sulfuric acid or the like; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc.; sulfonic acid salts prepared from methanesulfonic acid, ethanesulfonic acid, benzenesulfonic acid, p-toluenesulfonic acid, naphthalenesulfonic acid or the like; amino acid salts prepared from glycine, arginine, lysine, etc.; amine salts prepared from trimethylamine, triethylamine, ammonia, pyridine, picoline, etc.; and the like, but types of salts meant in the present invention are not limited to those listed salts.

In the present invention, preferable salts may include hydrochloric acid, trifluoroacetic acid, citric acid, bromic acid, maleic acid, phosphoric acid, sulfuric acid, tartaric acid, etc.

As one example, the pharmaceutically acceptable salt of the present invention may be a salt of compound 1 in the present specification.

The 1,3,4-oxadiazole triazole compound of the present invention may include at least one asymmetric carbon, and thus may be present as a racemate, racemic mixture, single enantiomer, mixture of diastereomers and respective diastereomers thereof. Such isomers of the compound represented by formula I may be separated by splitting according to the related art, for example, with a column chromatography, HPLC or the like. Alternatively, respective stereoisomers of the compound represented by formula (I) can be stereospecifically synthesized with a known arrangement of optically pure starting materials and/or reagents.

In the present invention, “stereoisomer” may include a diastereomer and an optical isomer, in which the optical isomer may include not only an enantiomer, but also a mixture of the enantiomer and even a racemate.

(9) The 1,3,4-oxadiazole triazole compound according to the present invention may be any one selected from the compounds shown in table 1 below.

TABLE 1

| Com- | |
|---|---|
| pound | |
| No. | Structure |
| 1 | 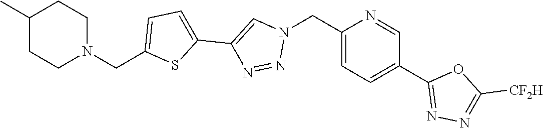 [SMILES: CC1CCN(Cc2ccc(-c3cn(Cc4ccc(-c5nnc(C(F)F)o5)cn4)nn3)s2)CC1] |
| 2 | 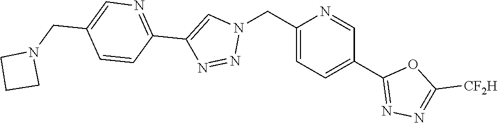 [SMILES: FC(F)c1nnc(-c2ccc(Cn3cc(-c4ccc(CN5CCC5)cn4)nn3)nc2)o1] |
| 3 | 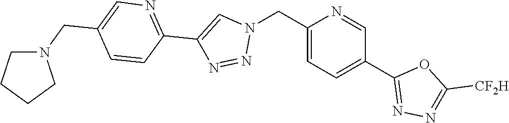 [SMILES: FC(F)c1nnc(-c2ccc(Cn3cc(-c4ccc(CN5CCCC5)cn4)nn3)nc2)o1] |
| 4 | 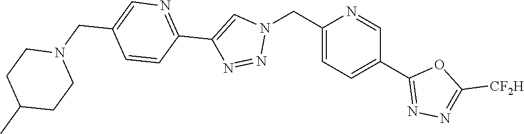 [SMILES: CC1CCN(Cc2ccc(-c3cn(Cc4ccc(-c5nnc(C(F)F)o5)cn4)nn3)nc2)CC1] |
| 5 | 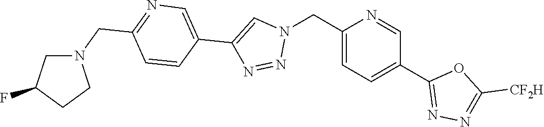 [SMILES: FC(F)c1nnc(-c2ccc(Cn3cc(-c4ccc(CN5CC[C@@H](F)C5)nc4)nn3)nc2)o1] |
| 6 | 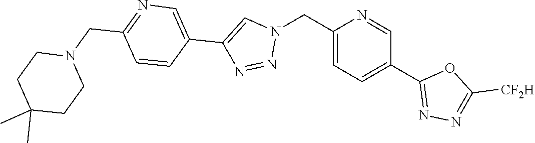 [SMILES: CC1(C)CCN(Cc2ccc(-c3cn(Cc4ccc(-c5nnc(C(F)F)o5)cn4)nn3)cn2)CC1] |
| 7 | 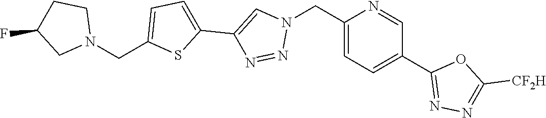 [SMILES: FC(F)c1nnc(-c2ccc(Cn3cc(-c4ccc(CN5CC[C@H](F)C5)s4)nn3)nc2)o1] |
| 8 | 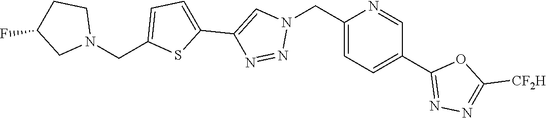 [SMILES: FC(F)c1nnc(-c2ccc(Cn3cc(-c4ccc(CN5CC[C@@H](F)C5)s4)nn3)nc2)o1] |
| 9 | 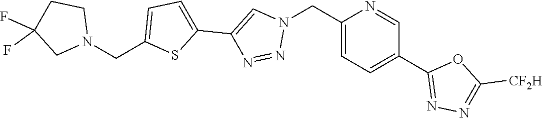 [SMILES: FC(F)c1nnc(-c2ccc(Cn3cc(-c4ccc(CN5CCC(F)(F)C5)s4)nn3)nc2)o1] |
| 10 | 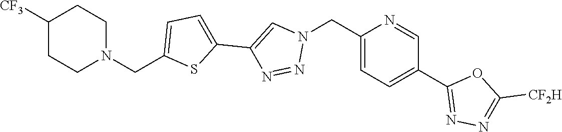 [SMILES: FC(F)c1nnc(-c2ccc(Cn3cc(-c4ccc(CN5CCC(C(F)(F)F)CC5)s4)nn3)nc2)o1] |
| 11 | 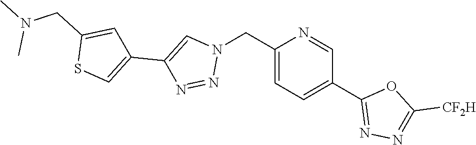 [SMILES: CN(C)Cc1cc(-c2cn(Cc3ccc(-c4nnc(C(F)F)o4)cn3)nn2)cs1] |
| 12 | 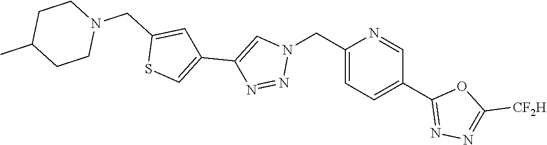 [SMILES: CC1CCN(Cc2cc(-c3cn(Cc4ccc(-c5nnc(C(F)F)o5)cn4)nn3)cs2)CC1] |
| 13 | 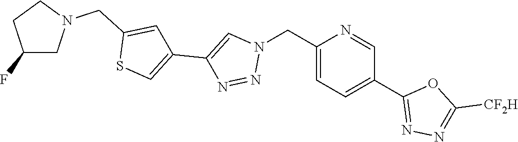 [SMILES: FC(F)c1nnc(-c2ccc(Cn3cc(-c4csc(CN5CC[C@H](F)C5)c4)nn3)nc2)o1] |
| 14 | 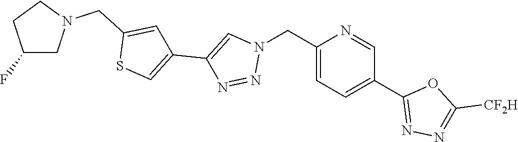 [SMILES: FC(F)c1nnc(-c2ccc(Cn3cc(-c4csc(CN5CC[C@@H](F)C5)c4)nn3)nc2)o1] |
| 15 | 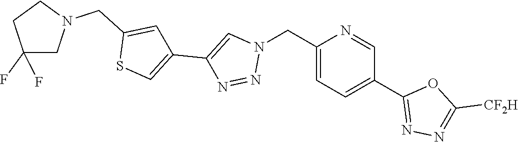 [SMILES: FC(F)c1nnc(-c2ccc(Cn3cc(-c4csc(CN5CCC(F)(F)C5)c4)nn3)nc2)o1] |
| 16 | 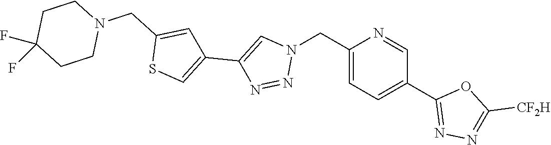 [SMILES: FC(F)c1nnc(-c2ccc(Cn3cc(-c4csc(CN5CCC(F)(F)CC5)c4)nn3)nc2)o1] |
| 17 | 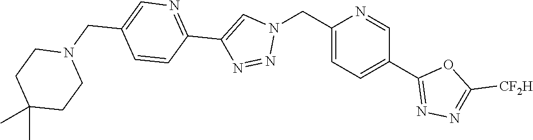 [SMILES: CC1(C)CCN(Cc2ccc(-c3cn(Cc4ccc(-c5nnc(C(F)F)o5)cn4)nn3)nc2)CC1] |
| 18 | 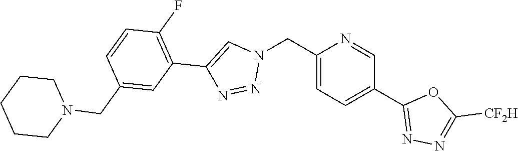 [SMILES: Fc1ccc(CN2CCCCC2)cc1-c1cn(Cc2ccc(-c3nnc(C(F)F)o3)cn2)nn1] |
| 19 | 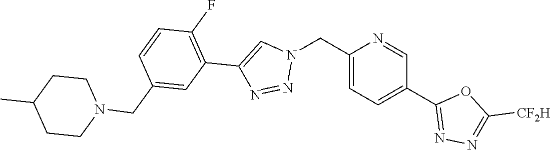 [SMILES: CC1CCN(Cc2ccc(F)c(-c3cn(Cc4ccc(-c5nnc(C(F)F)o5)cn4)nn3)c2)CC1] |
| 20 | 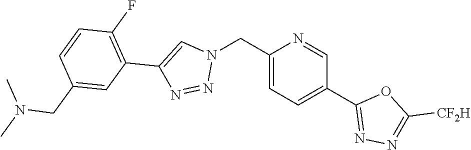 [SMILES: CN(C)Cc1ccc(F)c(-c2cn(Cc3ccc(-c4nnc(C(F)F)o4)cn3)nn2)c1] |
| 21 | 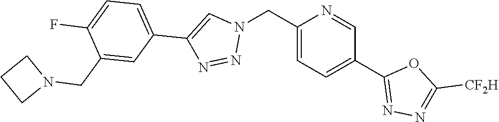 [SMILES: Fc1ccc(-c2cn(Cc3ccc(-c4nnc(C(F)F)o4)cn3)nn2)cc1CN1CCC1] |
| 22 | 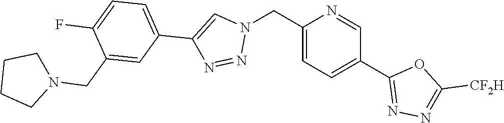 [SMILES: Fc1ccc(-c2cn(Cc3ccc(-c4nnc(C(F)F)o4)cn3)nn2)cc1CN1CCCC1] |
| 23 | 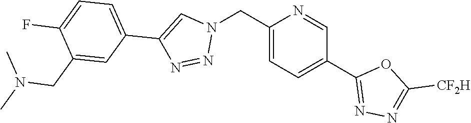 [SMILES: CN(C)Cc1cc(-c2cn(Cc3ccc(-c4nnc(C(F)F)o4)cn3)nn2)ccc1F] |
| 24 | 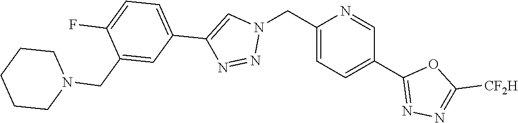 [SMILES: Fc1ccc(-c2cn(Cc3ccc(-c4nnc(C(F)F)o4)cn3)nn2)cc1CN1CCCCC1] |
| 25 | 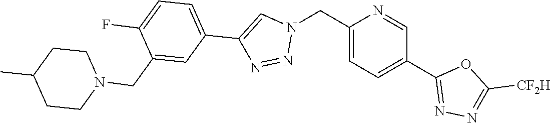 [SMILES: CC1CCN(Cc2cc(-c3cn(Cc4ccc(-c5nnc(C(F)F)o5)cn4)nn3)ccc2F)CC1] |
| 26 | 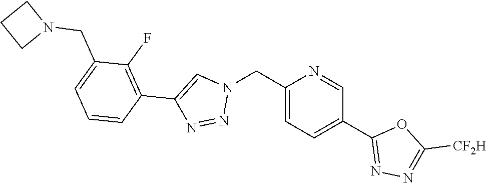 [SMILES: Fc1c(CN2CCC2)cccc1-c1cn(Cc2ccc(-c3nnc(C(F)F)o3)cn2)nn1] |
| 27 | 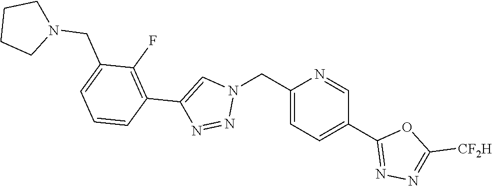 [SMILES: Fc1c(CN2CCCC2)cccc1-c1cn(Cc2ccc(-c3nnc(C(F)F)o3)cn2)nn1] |
| 28 | 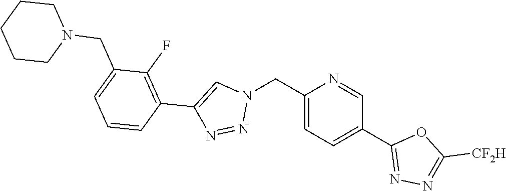 [SMILES: Fc1c(CN2CCCCC2)cccc1-c1cn(Cc2ccc(-c3nnc(C(F)F)o3)cn2)nn1] |
| 29 | 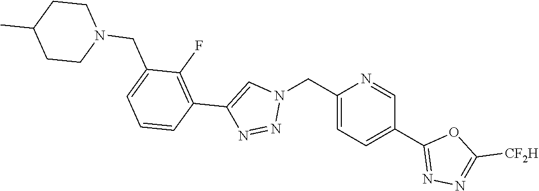 [SMILES: CC1CCN(Cc2cccc(-c3cn(Cc4ccc(-c5nnc(C(F)F)o5)cn4)nn3)c2F)CC1] |
| 30 | 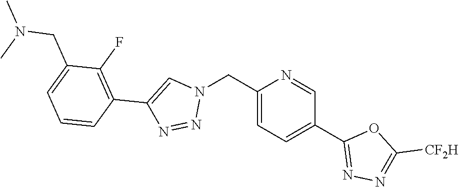 [SMILES: CN(C)Cc1cccc(-c2cn(Cc3ccc(-c4nnc(C(F)F)o4)cn3)nn2)c1F] |
| 31 | 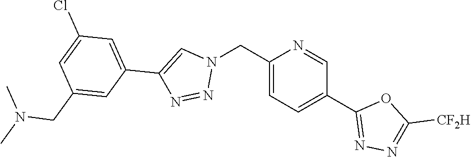 [SMILES: CN(C)Cc1cc(Cl)cc(-c2cn(Cc3ccc(-c4nnc(C(F)F)o4)cn3)nn2)c1] |
| 32 | 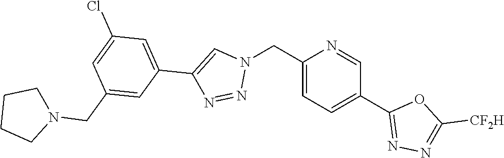 [SMILES: FC(F)c1nnc(-c2ccc(Cn3cc(-c4cc(Cl)cc(CN5CCCC5)c4)nn3)nc2)o1] |
| 33 | 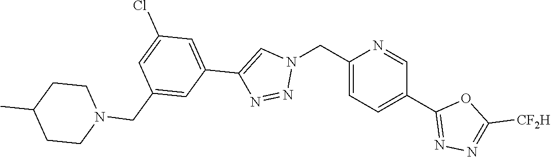 [SMILES: CC1CCN(Cc2cc(Cl)cc(-c3cn(Cc4ccc(-c5nnc(C(F)F)o5)cn4)nn3)c2)CC1] |
| 34 | 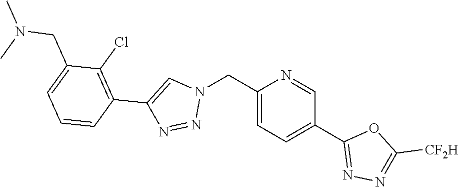 [SMILES: CN(C)Cc1cccc(-c2cn(Cc3ccc(-c4nnc(C(F)F)o4)cn3)nn2)c1Cl] |
| 35 | 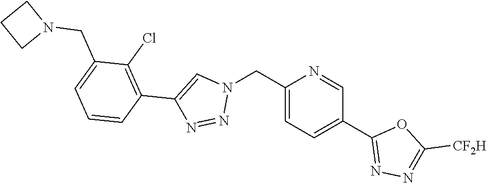 [SMILES: FC(F)c1nnc(-c2ccc(Cn3cc(-c4cccc(CN5CCC5)c4Cl)nn3)nc2)o1] |
| 36 | 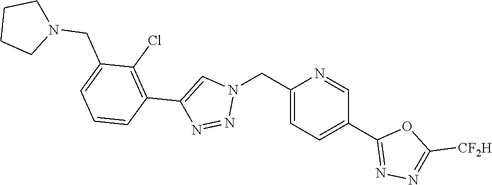 [SMILES: FC(F)c1nnc(-c2ccc(Cn3cc(-c4cccc(CN5CCCC5)c4Cl)nn3)nc2)o1] |
| 37 | 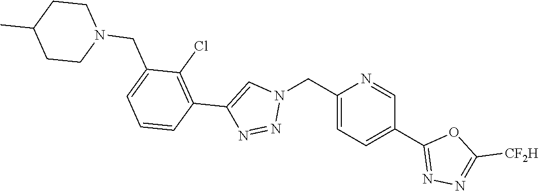 [SMILES: CC1CCN(Cc2cccc(-c3cn(Cc4ccc(-c5nnc(C(F)F)o5)cn4)nn3)c2Cl)CC1] |
| 38 | 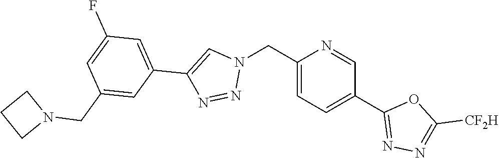 [SMILES: Fc1cc(CN2CCC2)cc(-c2cn(Cc3ccc(-c4nnc(C(F)F)o4)cn3)nn2)c1] |
| 39 | 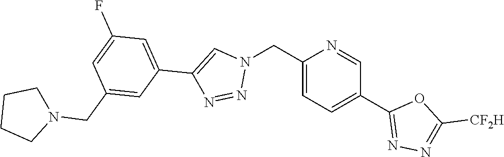 [SMILES: Fc1cc(CN2CCCC2)cc(-c2cn(Cc3ccc(-c4nnc(C(F)F)o4)cn3)nn2)c1] |
| 40 | 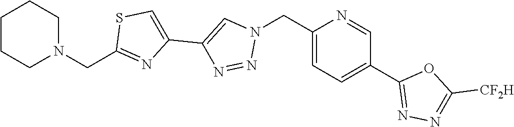 [SMILES: FC(F)c1nnc(-c2ccc(Cn3cc(-c4csc(CN5CCCCC5)n4)nn3)nc2)o1] |
| 41 | 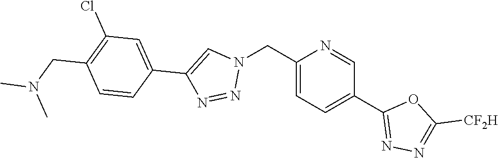 [SMILES: CN(C)Cc1ccc(-c2cn(Cc3ccc(-c4nnc(C(F)F)o4)cn3)nn2)cc1Cl] |
| 42 | 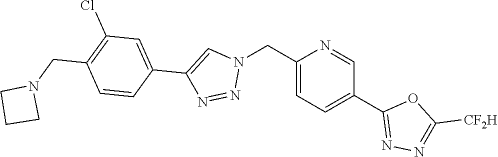 [SMILES: FC(F)c1nnc(-c2ccc(Cn3cc(-c4ccc(CN5CCC5)c(Cl)c4)nn3)nc2)o1] |

### Method for Preparing Compound of Formula I

A preferable method for preparing the 1,3,4-oxadiazole triazole compound according to the present invention, stereoisomers thereof or pharmaceutically acceptable salts thereof may follow reaction formulas 1 and 2, and even a preparation method modified at a level apparent to those skilled in the art may be also included therein.

Hereinafter, in reaction formulas 1 and 2, those which are represented by the same symbols as those of formula I and are not described in detail may be the same as those defined in formula I, and thus redundant descriptions are omitted.

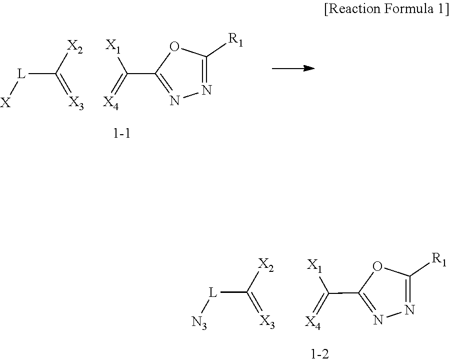

According to above reaction formula 1, compound 1-2 is synthesized through a reaction in which a halide portion of compound 1-1 is substituted with an azide. In above reaction scheme 1, X may refer to halide.

Compound 1-2 may be used in the synthesis of all compounds having a triazole scaffold.

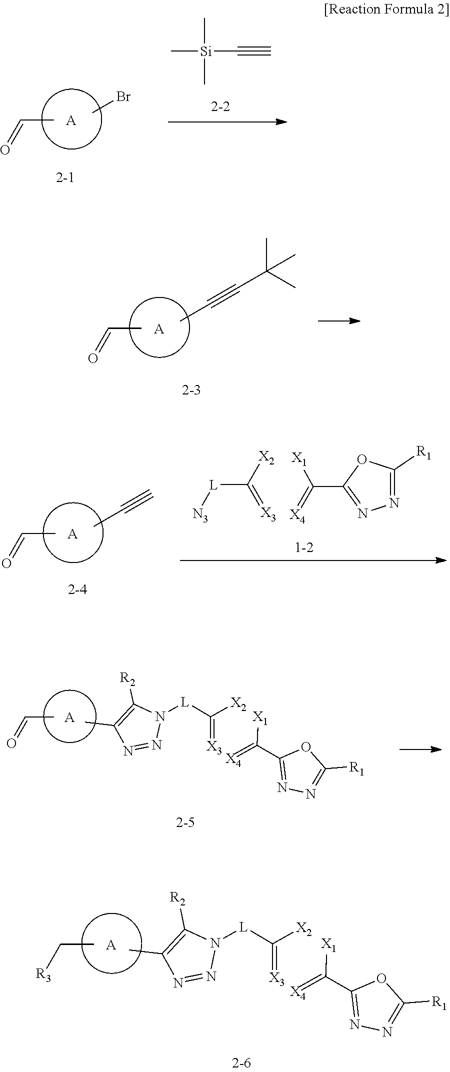

In above reaction formula 2, Ring A represented by

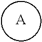

in each compound of reaction formula 2 may be C6-C12 aryl (in which at least one H of C6-C12 aryl is substituted with halogen) or may be 5- to 6-membered heteroaryl, in which R₃ may be —NR₄R₅ (in which R₄ and R₅ are each independently H or C1-C5alkyl) or

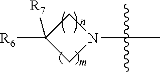

(in which R₆ and R₇ are each independently H, halogen, C1-C6 alkyl, or C1-C6 haloalkyl, and n and m are each independently 1 or 2).

According to above reaction formula 2, compound 2-3 having a trimethyl silane protecting group may be prepared through a C—C coupling (Sonogashira coupling) between halide compound 2-1 and compound 2-2 having a triple bond, after which compound 2-4 having an aldehyde structure may be prepared by removing the trimethyl silane protecting group.

Compound 2-5 having a triazol structure may be prepared through a click reaction between compound 2-4 and compound 1-2, after which compound 2-6 may be prepared through a reductive amination reaction.

The 1,3,4-oxadiazole triazole compounds according to the present invention may be prepared according to reaction formulas 1 and 2 described above.

Histone deacetylase 6-mediated diseases may include cancer, inflammatory disease, autoimmune disease, neurological or degenerative neurological disease, specifically, lung cancer, colon cancer, breast cancer, prostate cancer, liver cancer, brain cancer, ovarian cancer, gastric cancer, skin cancer, pancreatic cancer, glioma, glioblastoma carcinoma, leukemia, lymphoma, multiple myeloma, solid cancer, Wilson's disease, spinocerebellar ataxia, prion disease, Parkinson's disease, Huntington's disease, amyotrophic lateral sclerosis, amyloidosis, Alzheimer's disease, alcoholic liver disease, spinal muscular atrophy, rheumatoid arthritis or osteoarthritis, in addition to symptoms or diseases related to abnormal functions of histone deacetylase.

Examples of histone deacetylase-mediated diseases may include infectious diseases, neoplasm, endocrinopathy, nutritional and metabolic diseases, mental and behavioral disorders, neurological diseases, eye and ocular adnexal diseases, circulatory diseases, respiratory diseases, digestive troubles, skin and subcutaneous tissue diseases, musculoskeletal system and connective tissue diseases, or teratosis, deformities and chromosomal aberration.

The endocrinopathy, nutritional and metabolic diseases may be Wilson's disease, amyloidosis or diabetes, the mental and behavioral disorders may be depression or Rett syndrome, and the neurological diseases may be central nervous system atrophy, neurodegenerative disease, movement disorder, neuropathy, motor neuron disease or central nervous system demyelinating disease, the eye and ocular adnexal diseases may be uveitis, the skin and subcutaneous tissue diseases may be psoriasis, the musculoskeletal system and connective tissue diseases may be rheumatoid arthritis, osteoarthritis or systemic lupus erythematosus, the teratosis, deformities and chromosomal aberration may be autosomal dominant polycystic kidney disease, the infectious disease may be prion disease, the neoplasm may be benign tumor or malignant tumor, the circulatory disease may be atrial fibrillation or stroke, the respiratory disease may be asthma, and the digestive troubles may be alcoholic liver disease, inflammatory bowel disease, Crohn's disease or ulcerative bowel disease.

Said pharmaceutically acceptable salts may be the same as described in the pharmaceutically acceptable salts of 1,3,4-oxadiazole triazole compound according to the present invention.

For administration, the pharmaceutical composition of the present invention may further include at least one type of a pharmaceutically acceptable carrier, in addition to the 1,3,4-oxadiazole triazole compound, stereoisomers thereof or pharmaceutically acceptable salts thereof. In this case, the pharmaceutically acceptable carrier to be used may include saline solution, sterilized water, Ringer's solution, buffered saline, dextrose solution, maltodextrin solution, glycerol, ethanol and a mixture of at least one ingredient thereof, and may include the addition of other conventional additives such as antioxidant, buffer solution, bacteriostatic agent, etc., if needed. In addition, diluent, dispersing agent, surfactant, binder and lubricant may be further added to be formulated into injectable formulations such as aqueous solution, suspension, emulsion, etc., pill, capsule, granule or tablet. Thus, the composition of the present invention may be patch, liquid medicine, pill, capsule, granule, tablet, suppository, etc. The preparations may be prepared according to a conventional method used for formulation in the art or a method disclosed in Remington's Pharmaceutical Science (latest edition), Mack Publishing Company, Easton PA, and the composition may be formulated into various preparations depending on each disease or ingredient.

The composition of the present invention may be orally or parenterally administered (for example, applied intravenously, subcutaneously, intraperitoneally or locally) according to a targeted method, in which a dosage thereof may vary in a range thereof depending on a patient's weight, age, gender, health condition and diet, an administration time, an administration method, an excretion rate, a severity of a disease and the like. A daily dosage of the compound represented by formula I of the present invention may be about 1 to 1000 mg/kg, preferably 5 to 100 mg/kg, and may be administered at one time a day or several times a day by dividing the daily dosage of the compound.

In addition to the compound represented by formula I described above or the 1,3,4-oxadiazole triazole compound including the compound listed in table 1, stereoisomers thereof or pharmaceutically acceptable salts thereof, the pharmaceutical composition of the present invention may further include at least one active ingredient which shows the same or similar medicinal effects.

The present invention provides a method for preventing or treating histone deacetylase 6-mediated diseases, including administering a therapeutically effective amount of the compound represented by formula I described above or the 1,3,4-oxadiazole triazole compound including the compound listed in table 1, stereoisomers thereof or pharmaceutically acceptable salts thereof.

As used herein, the term “therapeutically effective amount” may refer to an amount of the compound represented by formula I described above or the 1,3,4-oxadiazole triazole compound including the compound listed in table 1, which is effective in preventing or treating histone deacetylase 6-mediated diseases.

In addition, the present invention provides a method for selectively inhibiting HDAC6 by administering the compound represented by formula I described above or the 1,3,4-oxadiazole triazole compound including the compound listed in table 1, stereoisomers thereof or pharmaceutically acceptable salts thereof into mammals including humans.

The method for preventing or treating histone deacetylase 6-mediated diseases according to the present invention may include not only dealing with the diseases per se before expression of symptoms, but also inhibiting or avoiding such symptoms by administering the compound represented by formula I described above or the 1,3,4-oxadiazole triazole compound including the compound listed in table 1. In managing the diseases, a preventive or therapeutic dose of a certain active ingredient may vary depending on a nature and severity of the diseases or conditions and a route of administering the active ingredient. A dose and a frequency thereof may vary depending on an individual patient's age, weight and reactions. A suitable dose and usage may be easily selected by those skilled in the art, naturally considering such factors. In addition, the method for preventing or treating histone deacetylase 6-mediated diseases of the present invention may further include administering a therapeutically effective amount of an additional active agent, which is helpful in treating the diseases, along with the compound represented by formula I described above or the 1,3,4-oxadiazole triazole compound including the compound listed in table 1, in which the additional active agent may show a synergy effect or an adjuvant effect together with the compound of above formula I.

The present invention also provides a use of the compound represented by formula I described above or the 1,3,4-oxadiazole triazole compound including the compound listed in table 1, stereoisomers thereof or pharmaceutically acceptable salts thereof in the manufacture of a medicament for preventing or treating histone deacetylase 6-mediated diseases. The compound represented by formula I described above or the 1,3,4-oxadiazole triazole compound including the compound listed in table 1 in the manufacture of a medicament may be combined with an acceptable adjuvant, diluent, carrier, etc., and may be prepared into a complex agent together with other active agents, thus having a synergy action.

Matters mentioned in the use, composition and therapeutic method of the present invention may be equally applied, if not contradictory to each other.

### Advantageous Effects

According to the present invention, the 1,3,4-oxadiazole triazole compound, stereoisomers thereof or pharmaceutically acceptable salts thereof can selectively inhibit HDAC6, and thus have a remarkably excellent effect of preventing or treating histone deacetylase 6 activity-related diseases.

### MODE FOR INVENTION

### Example 1: Synthesis of Compound 1, 2-(difluoromethyl)-5-(6-((4-(5-((4-methylpiperidin-1-yl)methyl)thiophen-2-yl)-1H-1,2,3-triazol-1-yl)methyl)pyridin-3-yl)-1,3,4-oxadiazole

### [Step 1] Synthesis of 2-(6-(azidomethyl)pyridin-3-yl)-5-(difluoromethyl)-1,3,4-oxadiazole

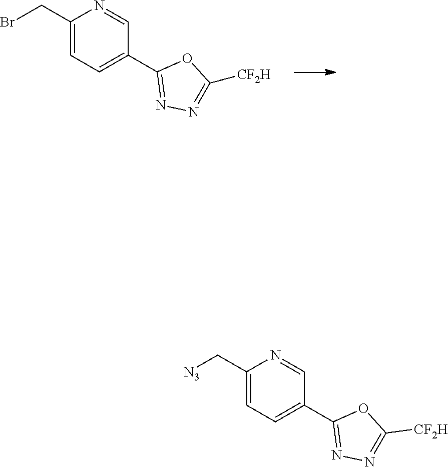 [SMILES: FC(F)c1nnc(-c2ccc(CBr)nc2)o1 ; [N-]=[N+]=NCc1ccc(-c2nnc(C(F)F)o2)cn1]

2-(6-(bromomethyl)pyridin-3-yl)-5-(difluoromethyl)-1,3,4-oxadiazole (1.000 g, 3.447 mmol) was dissolved in N,N-dimethylformamide (10 mL) at room temperature, after which sodium azide (0.224 g, 3.447 mmol) was added to the resulting solution and stirred at 40° C. for two hours, and then a reaction was finished by lowering a temperature to room temperature. Water was poured into the reaction mixture and extracted with dichloromethane. An organic layer was washed with saturated aqueous solution of sodium chloride, dehydrated with anhydrous sodium sulfate, filtered, and concentrated under reduced pressure. The resulting concentrate was purified via column chromatography (SiO₂, 24 g cartridge; ethyl acetate/hexane=0% to 50%) and concentrated to obtain 2-(6-(azidomethyl)pyridin-3-yl)-5-(difluoromethyl)-1,3,4-oxadiazole (0.800 g, 92.0%) in a yellow solid form.

### [Step 2] Synthesis of 5-((trimethylsilyl)ethynyl)thiophen-2-carbaldehyde

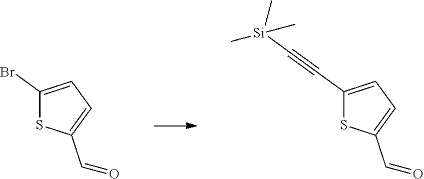

5-bromothiophen-2-carbaldehyde (0.622 mL, 5.210 mmol), bis(triphenylphosphine) palladium dichloride (0.073 g, 0.104 mmol), copper iodide (I/II, 0.010 g, 0.052 mmol) and diethylamine (10.778 mL, 104.199 mmol) were dissolved in tetrahydrofuran, after which trimethylsilyl acetylene (0.810 mL, 5.731 mmol) was added to the resulting solution at 0° C., stirred at the same temperature for 0.5 hour, and further stirred at room temperature for 18 hours. Solvent was removed from the reaction mixture under reduced pressure, after which water was poured into the resulting concentrate, and extracted with diethyl ether. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge;

dichloromethane/hexane=0% to 50%) and concentrated to obtain 5-((trimethylsilyl)ethynyl)thiophen-2-carbaldehyde (0.600 g, 55.3%) in a brown solid form.

### [Step 3] Synthesis of 5-ethynylthiophen-2-carbaldehyde

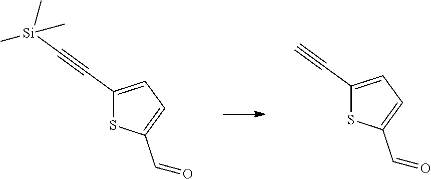

The 5-((trimethylsilyl)ethynyl)thiophen-2-carbaldehyde (0.550 g, 2.640 mmol) prepared in step 2 and potassium carbonate (1.094 g, 7.919 mmol) were dissolved in methanol (5 mL) at room temperature, after which the resulting solution was stirred at the same temperature for 18 hours. Water was poured into the reaction mixture and extracted with dichloromethane. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; ethyl acetate/hexane=0% to 20%) and concentrated to obtain 5-ethynylthiophen-2-carbaldehyde (0.300 g, 83.5%) in a light yellow solid form.

### [Step 4] Synthesis of 5-(1-((5-(5-(difluoromethyl)-1,3,4-oxadiazol-2-yl)pyridin-2-yl)methyl)-1H-1,2,3-triazol-4-yl)thiophen-2-carbaldehyde

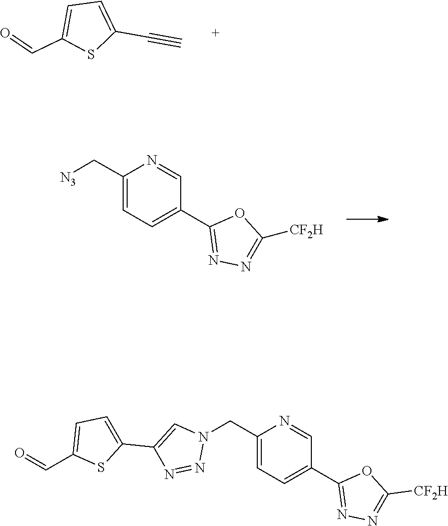

The 5-ethynylthiophen-2-carbaldehyde (0.250 g, 1.836 mmol) prepared in step 3 and 2-(6-(azidomethyl)pyridin-3-yl)-5-(difluoromethyl)-1,3,4-oxadiazole (0.463 g, 1.836 mmol) prepared in step 1 were dissolved in tert-butanol (5 mL)/water (5 mL) at room temperature, after which sodium ascorbate (1.00 M solution, 0.184 mL, 0.184 mmol) and copper sulfate (I/II, 0.50 M solution, 0.184 mL, 0.092 mmol) were added to the resulting solution and stirred at the same temperature for 18 hours. Saturated aqueous solution of ammonium chloride was poured into the reaction mixture and extracted with ethyl acetate. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; ethyl acetate/hexane=0% to 70%) and concentrated to obtain 5-(1-((5-(5-(difluoromethyl)-1,3,4-oxadiazol-2-yl)pyridin-2-yl)methyl)-1H-1,2,3-triazol-4-yl)thiophen-2-carbaldehyde (0.300 g, 42.1%) in a light yellow solid form.

### [Step 5] Synthesis of Compound 1

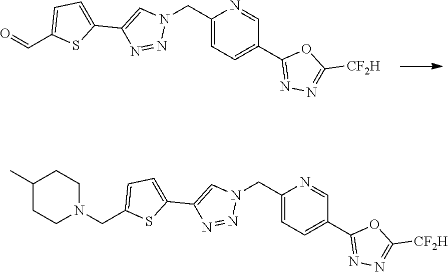 [SMILES: O=Cc1ccc(-c2cn(Cc3ccc(-c4nnc(C(F)F)o4)cn3)nn2)s1 ; CC1CCN(Cc2ccc(-c3cn(Cc4ccc(-c5nnc(C(F)F)o5)cn4)nn3)s2)CC1]

The 5-(1-((5-(5-(difluoromethyl)-1,3,4-oxadiazol-2-yl)pyridin-2-yl)methyl)-1H-1,2,3-triazol-4-yl)thiophen-2-carbaldehyde (0.040 g, 0.103 mmol) prepared in step 4 and 4-methylpiperidine (0.020 g, 0.206 mmol) were dissolved in dichloromethane (1 mL) at room temperature, after which sodium triacetoxyborohydride (0.109 g, 0.515 mmol) was added to the resulting solution and stirred at the same temperature for 18 hours. Saturated aqueous solution of sodium hydrogen carbonate was poured into the reaction mixture and extracted with dichloromethane. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; dichloromethane/methanol=100% to 20%) and concentrated to obtain 2-(difluoromethyl)-5-(6-((4-(5-((4-methylpiperidin-1-yl)methyl)thiophen-2-yl)-1H-1,2,3-triazol-1-yl)methyl)pyridin-3-yl)-1,3,4-oxadiazole (0.032 g, 65.9%) in a white solid form.

¹H NMR (400 MHZ, CD₃OD) δ 9.27 (d, J=1.6 Hz, 1H), 8.53 (dd, J=8.2, 2.2 Hz, 1H), 8.46 (s, 1H), 7.62 (d, J=8.4 Hz, 1H), 7.40 (d, J=3.6 Hz, 1H), 7.26 (t, J=51.4 Hz, 1H), 7.20 (d, J=3.2 Hz, 1H), 6.71 (s, 2H), 5.91 (s, 2H), 4.27 (s, 2H), 2.70 (t, J=12.6 Hz, 2H), 1.86 (d, J=12.8 Hz, 2H), 1.62 (s, 1H); LRMS (ES) m/z 472.3 (M⁺+1).

### Examples 7 to 16

Compounds 7 to 16 were synthesized through substantially the same process as the method for preparing compound 1, except for using a reactant of table 2 below instead of 4-methylpiperidine in step 5 of the method for preparing compound 1 according to Example 1.

TABLE 2

| | Compound | | Yield |
|---|---|---|---|
| Example | No. | Reactant | (%) |
| 7 | 7 | (S)-(+)-3-fluoropyrrolidine | 76 |
| 8 | 8 | (R)-(−)-3-fluoropyrrolidine | 76 |
| 9 | 9 | 3,3-difluoropyrrolidine | 79 |
| 10 | 10 | 4-(trifluoromethyl)piperidine | 62 |
| 11 | 11 | Dimethylamine | 58 |
| 12 | 12 | 4-methylpiperidine | 56 |
| 13 | 13 | (S)-(+)-3-fluoropyrrolidine | 44 |
| 14 | 14 | (R)-(−)-3-fluoropyrrolidine | 42 |
| 15 | 15 | 3,3-difluoropyrrolidine | 34 |
| 16 | 16 | 4,4-difluoropiperidine | 37 |

### Example 2: Synthesis of Compound 2, 2-(6-((4-(5-(azetidin-1-ylmethyl)pyridin-2-yl)-1H-1,2,3-triazol-1-yl)methyl)pyridin-3-yl)-5-(difluoromethyl)-1,3,4-oxadiazole

### [Step 1] Synthesis of 6-((trimethylsilyl)ethynyl)nicotinaldehyde

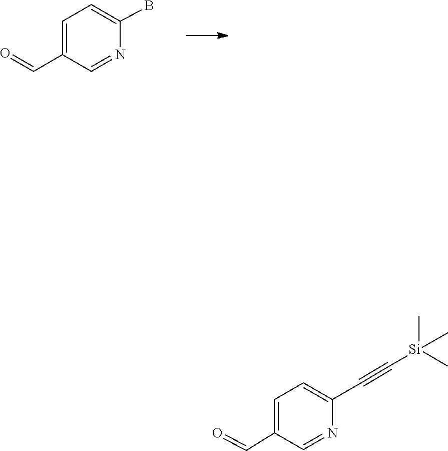

6-bromonicotinaldehyde (1.000 g, 5.376 mmol), bis(triphenylphosphin)palladium dichloride (0.151 g, 0.215 mmol), copper iodide (I/II, 0.102 g, 0.538 mmol) and 4,5-bis(diphenylphosphino)-9,9-diphenylxanthene (Xantphos, 0.124 g, 0.215 mmol) were dissolved in triethylamine (15 mL), after which trimethylsilyl acetylene (0.836 mL, 5.914 mmol) was added to the resulting solution at room temperature and stirred at the same temperature for 18 hours. The reaction mixture was filtered via a celite pad to remove a solid therefrom, after which solvent was removed from the resulting filtrate without the solid under reduced pressure. Then, the resulting concentrate was purified via column chromatography (SiO₂, 24 g cartridge; ethyl acetate/hexane=0% to 50%) and concentrated to obtain 6-((trimethylsilyl)ethynyl)nicotinaldehyde (0.400 g, 36.6%) in a light brown solid form.

### [Step 2] Synthesis of 6-ethynylnicotinaldehyde

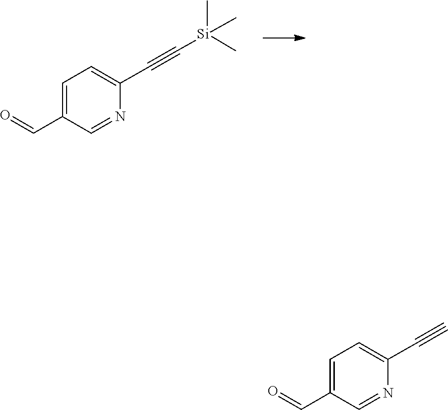

The 6-((trimethylsilyl)ethynyl)nicotinaldehyde (0.370 g, 1.820 mmol) prepared in step 1 and potassium carbonate (0.755 g, 5.459 mmol) were dissolved in methanol (5 mL) at room temperature, after which the resulting solution was stirred at the same temperature for 18 hours. Water was poured into the reaction mixture and extracted with dichloromethane. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; ethyl acetate/hexane=0% to 40%) and concentrated to obtain 6-ethynylnicotinaldehyde (0.200 g, 83.8%) in a beige solid form.

### [Step 3] Synthesis of 6-(1-((5-(5-(difluoromethyl)-1,3,4-oxadiazol-2-yl)pyridin-2-yl)methyl)-1H-1,2,3-triazol-4-yl)nicotinaldehyde

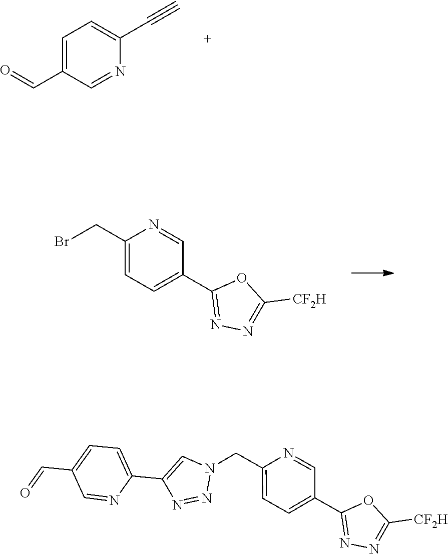

The 6-ethynylnicotinaldehyde (0.100 g, 0.763 mmol) prepared in step 2 and 2-(6-(azidomethyl)pyridin-3-yl)-5-(difluoromethyl)-1,3,4-oxadiazole (0.192 g, 0.763 mmol) prepared in step 1 of example 1 were dissolved in tert-butanol (2 mL)/water (2 mL) at room temperature, after which sodium ascorbate (1.00 M solution, 0.076 mL, 0.076 mmol) and copper sulfate (I/II, 1.00 M solution, 0.038 mL, 0.038 mmol) were added to the resulting solution and stirred at the same temperature for 18 hours. Saturated aqueous solution of ammonium chloride was poured into the reaction mixture and extracted with ethyl acetate. An organic layer was washed with saturated aqueous solution of sodium chloride, dehydrated with anhydrous sodium sulfate, filtered, and concentrated under reduced pressure. The resulting concentrate was purified via column chromatography (SiO₂, 12 g cartridge; ethyl acetate/hexane=0% to 50%) and concentrated to obtain 6-(1-((5-(5-(difluoromethyl)-1,3,4-oxadiazol-2-yl)pyridin-2-yl)methyl)-1H-1,2,3-triazol-4-yl)nicotinaldehyde (0.180 g, 61.6%) in a light yellow solid form.

### [Step 4] Synthesis of Compound 2

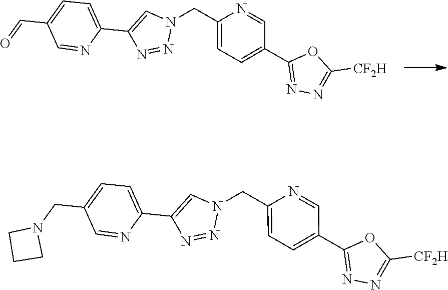 [SMILES: O=Cc1ccc(-c2cn(Cc3ccc(-c4nnc(C(F)F)o4)cn3)nn2)nc1 ; FC(F)c1nnc(-c2ccc(Cn3cc(-c4ccc(CN5CCC5)cn4)nn3)nc2)o1]

The 6-(1-((5-(5-(difluoromethyl)-1,3,4-oxadiazol-2-yl)pyridin-2-yl)methyl)-1H-1,2,3-triazol-4-yl)nicotinaldehyde (0.040 g, 0.104 mmol) prepared in step 3 and azetidine hydrochloride (0.020 g, 0.209 mmol) were dissolved in dichloromethane (1 mL) at room temperature, after which sodium triacetoxyborohydride (0.111 g, 0.522 mmol) was added to the resulting solution and stirred at the same temperature for 18 hours. Saturated aqueous solution of sodium hydrogen carbonate was poured into the reaction mixture and extracted with dichloromethane. An organic layer was washed with saturated aqueous solution of sodium chloride, dehydrated with anhydrous sodium sulfate, filtered, and concentrated under reduced pressure. The resulting concentrate was purified via column chromatography (SiO₂, 4 g cartridge; dichloromethane/methanol=100% to 80%) and concentrated to obtain 2-(6-((4-(5-(azetidin-1-ylmethyl)pyridin-2-yl)-1H-1,2,3-triazol-1-yl)methyl)pyridin-3-yl)-5-(difluoromethyl)-1,3,4-oxadiazole (0.021 g, 47.4%) in a white solid form.

### Examples 3 to 6 and 17

Compounds 3 to 6 and 17 according to Examples 3 to 6 and 17 were each synthesized through substantially the same process as the method for preparing compound 2, except for using a reactant of table 3 below instead of azetidine in step 4 of the method for preparing compound 2 according to Example 2.

TABLE 3

| | Compound | | Yield |
|---|---|---|---|
| Example | No. | Reactant | (%) |
| 3 | 3 | Pyrrolidine | 55 |
| 4 | 4 | 4-methylpiperidine | 60 |
| 5 | 5 | (R)-(−)-3-fluoropyrrolidine | 61 |
| 6 | 6 | 4,4-dimethylpiperidine | 56 |
| 17 | 17 | 4,4-dimethylpiperidine | 54 |

### Examples 18 to 39, 41 and 42

A reaction was made between a product obtained by reacting reactant 1 of table 4 below with 2-(6-(azidomethyl)pyridin-3-yl)-5-(difluoromethyl)-1,3,4-oxadiazole instead of 6-ethynylnicotinaldehyde in step 3 of the preparation method of compound 2 according to example 2, and reactant 2 of table 4 below through substantially the same process as in step 4 of example 2, thereby preparing compounds 18 to 39, 41, and 42 according to examples 18 to 39, 41, and 42, respectively.

TABLE 4

| | Compound | | | Yield |
|---|---|---|---|---|
| Example | No. | Reactant 1 | Reactant 2 | (%) |
| 18 | 18 | 3-ethynyl-5-fluorobenzaldehyde | Piperidine | 36 |
| 19 | 19 | 3-ethynyl-5-fluorobenzaldehyde | 4-methylpiperidine | 36 |
| 20 | 20 | 3-ethynyl-5-fluorobenzaldehyde | Dimethylamine hydrochloride | 54 |
| 21 | 21 | 5-ethynyl-2-fluorobenzaldehyde | Azetidine | 52 |
| 22 | 22 | 5-ethynyl-2-fluorobenzaldehyde | Pyrrolidine | 52 |
| 23 | 23 | 5-ethynyl-2-fluorobenzaldehyde | Dimethylamine | 44 |
| 24 | 24 | 5-ethynyl-2-fluorobenzaldehyde | Piperidine | 45 |
| 25 | 25 | 5-ethynyl-2-fluorobenzaldehyde | 4-methylpiperidine | 52 |
| 26 | 26 | 3-ethynyl-2-fluorobenzaldehyde | Azetidine | 38 |
| 27 | 27 | 3-ethynyl-2-fluorobenzaldehyde | Pyrrolidine | 33 |
| 28 | 28 | 3-ethynyl-2-fluorobenzaldehyde | Piperidine | 38 |
| 29 | 29 | 3-ethynyl-2-fluorobenzaldehyde | 4-methylpiperidine | 51 |
| 30 | 30 | 3-ethynyl-2-fluorobenzaldehyde | Dimethylamine hydrochloride | 47 |
| 31 | 31 | 3-chloro-5-ethynylbenzaldehyde | Dimethylamine | 56 |
| 32 | 32 | 3-chloro-5-ethynylbenzaldehyde | Pyrrolidine | 71 |
| 33 | 33 | 3-chloro-5-ethynylbenzaldehyde | 4-methylpiperidine | 15 |
| 34 | 34 | 2-chloro-3-ethynylbenzaldehyde | Dimethylamine | 55 |
| 35 | 35 | 2-chloro-3-ethynylbenzaldehyde | Azetidine | 68 |
| 36 | 36 | 2-chloro-3-ethynylbenzaldehyde | Pyrrolidine | 79 |
| 37 | 37 | 2-chloro-3-ethynylbenzaldehyde | 4-methylpiperidine | 93 |
| 38 | 38 | 3-ethynyl-5-fluorobenzaldehyde | Azetidine | 69 |
| 39 | 39 | 3-ethynyl-5-fluorobenzaldehyde | Pyrrolidine | 62 |
| 41 | 41 | 2-chloro-4-ethynylbenzaldehyde | Dimethylamine | 14 |
| 42 | 42 | 2-chloro-4-ethynylbenzaldehyde | Azetidine | 26 |

### Example 40: Synthesis of Compound 40, 2-(difluoromethyl)-5-(6 ((4-(2-(piperidin-1-ylmethyl)thiazol-4-yl)-1H-1,2,3-triazol-1-yl)methyl)pyridin-3-yl)-1,3,4-oxadiazole

A reaction was made between a product obtained by reacting 4-ethynylthiazol-2-carbaldehyde with 2-(6-(azidomethyl)pyridin-3-yl)-5-(difluoromethyl)-1,3,4-oxadiazole instead of 6-ethynylnicotinaldehyde in step 3 of the preparation method of compound 2 according to example 2, and piperidine through substantially the same process as in step 4 of example 2, thereby preparing compound 40 of example 40 (yield 61%).

Compounds 2 to 42 as final products obtained according to above examples 2 to 42 and analytical data therefor are shown in table 5 below.

TABLE 5

| | Compound | |
|---|---|---|
| Example | No. | Compound Name, ¹H-NMR, MS (ESI) |
| 2 | 2 | 2-(6-((4-(5-(azetidin-1-ylmethyl)pyridin-2-yl)-1H-1,2,3-triazol-1- |
| | | yl)methyl)pyridin-3-yl)-5-(difluoromethyl)-1,3,4-oxadiazole |
| | | ¹H NMR (400 MHz, CD₃OD) δ 9.19 (d, J = 1.2 Hz, 1H), 8.73 (s, 1H), 8.49~8.47 |
| | | (m, 2H), 8.00 (d, J = 8.0 Hz, 1H), 7.78 (dd, J = 8.0, 2.4 Hz, 1H), 7.58 |
| | | (t, J = 51.2 Hz, 1H), 7.56 (d, J = 7.6 Hz, 1H), 5.96 (s, 2H), 3.57 (s, 2H), 3.15 |
| | | (t, J = 7.0 Hz, 4H), 1.99 (t, J = 7.0 Hz, 2H); LRMS (ES) m/z 425.03 (M⁺ + 1) |
| 3 | 3 | 2-(difluoromethyl)-5-(6-((4-(5-(pyrrolidin-1-ylmethyl)pyridin-2-yl)-1H- |
| | | 1,2,3-triazol-1-yl)methyl)pyridin-3-yl)-1,3,4-oxadiazole |
| | | ¹H NMR (400 MHz, CD3OD) δ 9.20 (d, J = 1.6 Hz, 1H), 8.74 (s, 1H), 8.53 |
| | | (d, J = 1.6 Hz, 1H), 8.49 (dd, J = 8.0, 2.4 Hz, 1H), 8.02 (d, J = 7.6 Hz, 1H), |
| | | 7.83 (dd, J = 8.0, 2.0 Hz, 1H), 7.58 (t, J = 51.4 Hz, 1H), 7.56 (d, J = 8.4 Hz, |
| | | 1H), 5.97 (s, 2H), 3.64 (s, 2H), 2.50~2.24 (m, 4H), 1.74~1.70 (m, 4H); |
| | | LRMS (ES) m/z 438.88 (M⁺ + 1) |
| 4 | 4 | 2-(difluoromethyl)-5-(6-((4-(5-((4-methylpiperidin-1-yl)methyl)pyridin-2- |
| | | yl)-1H-1,2,3-triazol-1-yl)methyl)pyridin-3-yl)-1,3,4-oxadiazole |
| | | ¹H NMR (400 MHz, DMSO-d6) δ 9.19 (d, J = 1.6 Hz, J H), 8.74 (s, 1H), |
| | | 8.50 (s, 1H), 8.49 (dd, J = 8.2, 2.2 Hz, 1H), 8.02 (d, J = 8.0 Hz, 1H), 7.81 (dd, |
| | | J = 8.0, 2.0 Hz, 1H), 7.58 (t, J = 51.2 Hz, 1H), 7.56 (d, J = 8.0 Hz, 1H), 5.97 |
| | | (s, 2H), 3.50 (s, 2H), 2.78 (d, J = 11.2 Hz, 2H), 1.95 (dd, J = 11.6, 9.6 Hz, |
| | | 2H), 1.57 (d, J = 11.6 Hz, 2H), 1.40~1.28 (m, 1H), 1.19~1.12 (m, 2H), 0.89 |
| | | (d, J = 6.8 Hz, 3H); LRMS (ES) m/z 467.07 (M⁺ + 1) |
| 5 | 5 | (R)-2-(difluoromethyl)-5-(6-((4-(6-((3-fluoropyrrolidin-1-yl)methyl)pyridin- |
| | | 3-yl)-1H-1,2,3-triazol-1-yl)methyl)pyridin-3-yl)-1,3,4-oxadiazole |
| | | ¹H NMR (400 MHz, DMSO-d6) δ 9.20 (d, J = 2.0 Hz, 1H), 8.99 (d, J = 2.0 |
| | | Hz, 1H), 8.82 (s, 1H), 8.50 (dd, J = 8.2, 2.2 Hz, 1H), 8.23 (dd, J = 8.4, 2.4 |
| | | Hz, 1H), 7.60 (d, J = 8.4 Hz, 1H), 7.58 (t, J = 51.2 Hz, 1H), 7.52 (d, J = 8.0 |
| | | Hz, 1H), 5.96 (s, 2H), 5.31~5.30 (m, 0.5H), 5.20~5.14 (m, 0.5H), 3.77 (s, |
| | | 2H), 3.33~2.84 (m, 2H), 2.82~2.64 (m, 2H), 2.21~2.01 (m, 1H), |
| | | 1.96~1.84 (m, 1H); LRMS (ES) m/z 457.30 (M⁺ + 1) |
| 6 | 6 | 2-(difluoromethyl)-5-(6-((4-(6-((4,4-dimethylpiperidin-1-yl)methyl)pyridin- |
| | | 3-yl)-1H-1,2,3-triazol-1-yl)methyl)pyridin-3-yl)-1,3,4-oxadiazole |
| | | ¹H NMR (400 MHz, DMSO-d6) δ 9.20 (d, J = 2.0 Hz, 1H), 8.98 (s, 1H), 8.81 |
| | | (s, 1H), 8.50 (dd, J = 8.2, 2.2 Hz, 1H), 8.21 (dd, J = 8.0, 2.0 Hz, 1H), 7.59 (d, |
| | | J = 9.6 Hz, 1H), 7.58 (t, J = 51.2 Hz, 1H), 7.59 (d, J = 9.6 Hz, 1H), 7.52 (d, |
| | | J = 8.4 Hz, 1H), 5.96 (s, 2H), 3.62 (s, 2H), 2.40 (t, J = 5.2 Hz, 4H), 1.35 (t, |
| | | J = 5.6 Hz, 4H), 0.91 (s, 6H); LRMS (ES) m/z 481.29 (M⁺ + 1) |
| 7 | 7 | (S)-2-(difluoromethyl)-5-(6-((4-(5-((3-fluoropyrrolidin-1-yl)methyl)thiophen- |
| | | 2-yl)-1H-1,2,3-triazol-1-yl)methyl)pyridin-3-yl)-1,3,4-oxadiazole |
| | | ¹H NMR (400 MHz, DMSO-d6) δ 9.19 (s, 1H), 8.58 (s, 1H), 8.49 (dd, J = 8.2, |
| | | 2.2 Hz, 1H), 7.58 (t, J = 51.4 Hz, 1H), 7.57 (d, J = 10.4 Hz, 1H), 7.29 (d, |
| | | J = 3.6 Hz, 1H), 6.97 (d, J = 3.2 Hz, 1H), 5.91 (s, 2H), 5.31~5.29 (m, 0.5H), |
| | | 5.19~5.11 (m, 0.5H), 3.83 (s, 2H), 2.89~2.81 (m, 2H), 2.71~2.65 (m, 2H), |
| | | 2.20~2.09 (m, 2H); LRMS (ES) m/z 462.3 (M⁺ + 1) |
| 8 | 8 | (R)-2-(difluoromethyl)-5-(6-((4-(5-((3-fluoropyrrolidin-1-yl)methyl)thiophen- |
| | | 2-yl)-1H-1,2,3-triazol-1-yl)methyl)pyridin-3-yl)-1,3,4-oxadiazole |
| | | ¹H NMR (400 MHz, DMSO-d6) δ 9.19 (s, 1H), 8.58 (s, 1H), 8.49 (dd, J = 8.2, |
| | | 2.2 Hz, 1H), 7.58 (t, J = 51.4 Hz, 1H), 7.57 (d, J = 10.4 Hz, 1H), 7.29 (d, |
| | | J = 3.6 Hz, 1H), 6.97 (d, J = 3.2 Hz, 1H), 5.91 (s, 2H), 5.31~5.29 (m, 0.5H), |
| | | 5.19~5.11 (m, 0.5H), 3.83 (s, 2H), 2.89~2.81 (m, 2H), 2.71~2.65 (m, 2H), |
| | | 2.20~2.09 (m, 2H); LRMS (ES) m/z 462.3 (M⁺ + 1) |
| 9 | 9 | 2-(difluoromethyl)-5-(6-((4-(5-((3,3-difluoropyrrolidin-1-yl)methyl)thiophen- |
| | | 2-yl)-1H-1,2,3-triazol-1-yl)methyl)pyridin-3-yl)-1,3,4-oxadiazole |
| | | ¹H NMR (400 MHz, DMSO-d6) δ 9.19 (dd, J = 2.0, 0.8 Hz, 1H), 8.60 (s, |
| | | 1H), 8.49 (dd, J = 8.2, 2.2 Hz, 1H), 7.58 (t, J = 51.2 Hz, 1H), 7.57 (d, J = 8.8 |
| | | Hz, 1H), 7.31 (d, J = 3.2 Hz, 1H), 6.99 (d, J = 3.6 Hz, 1H), 5.92 (s, 2H), 3.85 |
| | | (s, 2H), 2.93 (t, J = 13.4 Hz, 2H), 2.77 (t, J = 7.0 Hz, 2H), 2.34~2.22 (m, |
| | | 2H); LRMS (ES) m/z 480.3 (M⁺ + 1) |
| 10 | 10 | 2-(difluoromethyl)-5-(6-((4-(5-((4-(trifluoromethyl)piperidin-1-yl)methyl)thiophen- |
| | | 2-yl)-1H-1,2,3-triazol-1-yl)methyl)pyridin-3-yl)-1,3,4-oxadiazole |
| | | ¹H NMR (400 MHz, DMSO-d6) δ 9.19 (d, J = 2.0 Hz, 1H), 8.58 (s, 1H), 8.49 |
| | | (dd, J = 8.2, 2.2 Hz, 1H), 7.58 (t, J = 51.2 Hz, 1H), 7.56 (d, J = 8.4 Hz, 1H), |
| | | 7.29 (d, J = 3.6 Hz, 1H), 6.96 (d, J = 3.6 Hz, 1H), 5.91 (s, 2H), 3.71 (s, 2H), |
| | | 2.97 (d, J = 11.6 Hz, 2H), 2.34~2.27 (m, 1H), 2.02 (t, J = 10.8 Hz, 2H), 1.80 |
| | | (d, J = 12.0 Hz, 2H), 1.50~1.44 (m, 2H); LRMS (ES) m/z 526.3 (M⁺ + 1) |
| 11 | 11 | 1-(4-(1-((5-(5-(difluoromethyl)-1,3,4-oxadiazol-2-yl)pyridin-2-yl)methyl)- |
| | | 1H-1,2,3-triazol-4-yl)thiophen-2-yl)-N,N-dimethylmethanamine |
| | | ¹H NMR (400 MHz, DMSO-d6) δ 9.19 (d, J = 2.4 Hz, 1H), 8.53 (s, 1H), 8.49 |
| | | (dd, J = 8.0, 2.4 Hz, 1H), 7.76 (d, J = 1.2 Hz, 1H), 7.58 (t, J = 51.2 Hz, 1H), |
| | | 7.75 (d, J = 8.4 Hz, 1H), 7.37 (s, 1H), 5.91 (s, 2H), 3.62 (s, 2H), 2.20 (s, 6H); |
| | | LRMS (ES) m/z 418.3 (M⁺ + 1) |
| 12 | 12 | 2-(difluoromethyl)-5-(6-((4-(5-((4-methylpiperidin-1-yl)methyl)thiophen-3- |
| | | yl)-1H-1,2,3-triazol-1-yl)methyl)pyridin-3-yl)-1,3,4-oxadiazole |
| | | ¹H NMR (400 MHz, DMSO-d6) δ 9.19 (d, J = 1.6 Hz, 1H), 8.52 (s, 1H), 8.49 |
| | | (dd, J = 8.3, 2.3 Hz, 1H), 7.73 (d, J = 1.2 Hz, 1H), 7.58 (t, J = 51.2 Hz, 1H), |
| | | 7.54 (d, J = 8.0 Hz, 1H), 7.35 (s, 1H), 5.91 (s, 2H), 3.66 (s, 2H), 2.84 (d, J = 11.2 |
| | | Hz, 2H), 1.96 (t, J = 10.6 Hz, 2H), 1.58 (d, J = 10.4 Hz, 2H), 1.33~1.30 |
| | | (m, 1H), 1.19~1.10 (m, 2H), 0.89 (d, J = 6.4 Hz, 3H); LRMS (ES) m/z 472.3 |
| | | (M⁺ + 1) |
| 13 | 13 | (S)-2-(difluoromethyl)-5-(6-((4-(5-((3-fluoropyrrolidin-1-yl)methyl)thiophen- |
| | | 3-yl)-1H-1,2,3-triazol-1-yl)methyl)pyridin-3-yl)-1,3,4-oxadiazole |
| | | ¹H NMR (400 MHz, DMSO-d6) δ 9.19 (d, J = 2.0 Hz, 1H), 8.53 (s, 1H), 8.49 |
| | | (dd, J = 8.2, 2.2 Hz, 1H), 7.76 (d, J = 1.2 Hz, 1H), 7.58 (t, J = 51.4 Hz, 1H), |
| | | 7.55 (d, J = 8.4 Hz, 1H), 7.38 (d, J = 6.8 Hz, 1H), 5.91 (s, 2H), 5.29~5.25 |
| | | (m, 0.5H), 5.16~5.12 (m, 0.5H), 3.84 (s, 2H), 2.89~2.89 (m, 2H), 2.74~2.62 |
| | | (m, 1H), 2.42~2.33 (m, 1H), 2.23~2.09 (m, 1H), 1.98~1.87 (m, 1H); |
| | | LRMS (ES) m/z 462.3 (M⁺ + 1) |
| 14 | 14 | (R)-2-(difluoromethyl)-5-(6-((4-(5-((3-fluoropyrrolidin-1-yl)methyl)thiophen- |
| | | 3-yl)-1H-1,2,3-triazol-1-yl)methyl)pyridin-3-yl)-1,3,4-oxadiazole |
| | | ¹H NMR (400 MHz, DMSO-d6) δ 9.19 (d, J = 2.0 Hz, 1H), 8.53 (s, 1H), 8.49 |
| | | (dd, J = 8.2, 2.2 Hz, 1H), 7.76 (d, J = 1.2 Hz, 1H), 7.58 (t, J = 51.4 Hz, 1H), |
| | | 7.55 (d, J = 8.4 Hz, 1H), 7.38 (d, J = 6.8 Hz, 1H), 5.91 (s, 2H), 5.29~5.25 |
| | | (m, 0.5H), 5.16~5.12 (m, 0.5H), 3.84 (s, 2H), 2.89~2.89 (m, 2H), 2.74~2.62 |
| | | (m, 1H), 2.42~2.33 (m, 1H), 2.23~2.09 (m, 1H), 1.98~1.87 (m, 1H); |
| | | LRMS (ES) m/z 462.3 (M⁺ + 1) |
| 15 | 15 | 2-(difluoromethyl)-5-(6-((4-(5-((3,3-difluoropyrrolidin-1-yl)methyl)thiophen- |
| | | 3-yl)-1H-1,2,3-triazol-1-yl)methyl)pyridin-3-yl)-1,3,4-oxadiazole |
| | | ¹H NMR (400 MHz, DMSO-d6) δ 9.20 (d, J = 2.4 Hz, 1H), 8.54 (s, 1H), 8.49 |
| | | (dd, J = 8.0, 2.0 Hz, 1H), 7.78 (d, J = 1.2 Hz, 1H), 7.58 (t, J = 51.2 Hz, 1H), |
| | | 7.55 (d, J = 8.4 Hz, 1H), 7.41 (s, 1H), 5.91 (s, 2H), 3.87 (s, 2H), 2.94 (t, |
| | | J = 13.4 Hz, 2H), 2.77 (t, J = 7.0 Hz, 2H), 2.33~2.22 (m, 2H); LRMS (ES) |
| | | m/z 480.3 (M⁺ + 1) |
| 16 | 16 | 2-(difluoromethyl)-5-(6-((4-(5-((4,4-difluoropiperidin-1-yl)methyl)thiophen- |
| | | 3-yl)-1H-1,2,3-triazol-1-yl)methyl)pyridin-3-yl)-1,3,4-oxadiazole |
| | | ¹H NMR (400 MHz, DMSO-d6) δ 9.19 (d, J = 2.0 Hz, 1H), 8.53 (s, 1H), 8.49 |
| | | (dd, J = 8.2, 2.2 Hz, 1H), 7.77 (d, J = 1.2 Hz, 1H), 7.58 (t, J = 51.4 Hz, 1H), |
| | | 7.55 (d, J = 8.0 Hz, 1H), 7.40 (s, 1H), 5.91 (s, 2H), 3.80 (s, 2H), 2.57~2.51 |
| | | (m, 4H), 2.05~1.93 (m, 4H); LRMS (ES) m/z 480.3 (M⁺ + 1) |
| 17 | 17 | 2-(difluoromethyl)-5-(6-((4-(5-((4,4-dimethylpiperidin-1-yl)methyl)pyridin- |
| | | 2-yl)-1H-1,2,3-triazol-1-yl)methyl)pyridin-3-yl)-1,3,4-oxadiazole |
| | | ¹H NMR (400 MHz, CD₃OD) δ 9.28 (d, J = 1.6 Hz, 1H), 8.61 (s, 1H), 8.53 |
| | | (dd, J = 7.4, 3.0 Hz, 1H), 8.09 (d, J = 8.4 Hz, 1H), 7.93 (d, J = 8.0 Hz, 1H), |
| | | 7.62 (d, J = 8.0 Hz, 1H), 7.62 (d, J = 8.0 Hz, 1H), 7.26 (t, J = 51.6 Hz, 1H), |
| | | 5.97 (s, 2H), 3.64 (s, 2H), 2.54~2.50 (m, 4H), 1.46 (t, J = 5.6 Hz, 4H), 0.97 |
| | | (s, 6H); LRMS (ES) m/z 482.0 (M⁺ + 1) |
| 18 | 18 | 2-(difluoromethyl)-5-(6-((4-(2-fluoro-5-(piperidin-1-ylmethyl)phenyl)-1H- |
| | | 1,2,3-triazol-1-yl)methyl)pyridin-3-yl)-1,3,4-oxadiazole |
| | | ¹H NMR (400 MHz, CDCl₃) δ 9.35 (d, J = 2.0 Hz, 1H), 8.41 (dd, J = 8.2, 2.2 |
| | | Hz, 1H), 8.24 (dd, J = 7.2, 2.0 Hz, 1H), 8.15 (s, 1H), 7.40-7.38 (m, 2H), |
| | | 7.14-7.09 (m, 1H), 7.08 (s, 0.2H), 6.96 (s, 0.5H), 6.83 (s, 0.2H), 5.86 (s, |
| | | 2H), 3.59 (s, 2H), 2.84 (brs, 4H), 1.66-1.62 (m, 4H), 1.47-1.46 (m, 2H); |
| | | LRMS (ES) m/z 470.53 (M⁺ + 1). |
| 19 | 19 | 2-(difluoromethyl)-5-(6-((4-(2-fluoro-5-((4-methylpiperidin-1- |
| | | yl)methyl)phenyl)-1H-1,2,3-triazol-1-yl)methyl)pyridin-3-yl)-1,3,4-oxadiazole |
| | | ¹H NMR (400 MHz, CDCl₃) δ 9.34 (d, J = 2.0 Hz, 1H), 8.41 (dd, J = 8.2, 2.2 |
| | | Hz, 1H), 8.24-8.22 (m, 1H), 8.15 (d, J = 3.5 Hz, 1H), 7.41-7.38 (m, 2H), |
| | | 7.14-7.11 (m, 1H), 7.08 (s, 0.2H), 6.96 (s, 0.5H), 6.83 (s, 0.2H), 5.85 (s, 2H), |
| | | 3.64 (s, 2H), 2.99-2.96 (m, 2H), 2.13-2.10 (m, 2H), 1.64-1.62 (m, 2H), |
| | | 1.41-1.27 (m, 3H), 0.93 (d, J = 10.4 Hz, 3H); LRMS (ES) m/z 484.52 (M⁺ + 1). |
| 20 | 20 | 1-(3-(1-((5-(5-(difluoromethyl)-1,3,4-oxadiazol-2-yl)pyridin-2-yl)methyl)- |
| | | 1H-1,2,3-triazol-4-yl)-4-fluorophenyl)-N,N-dimethylmethanamine |
| | | ¹H NMR (400 MHz, CDCl₃) δ 9.35 (d, J = 2.0 Hz, 1H), 8.42 (dd, J = 8.2, 2.2 |
| | | Hz, 1H), 8.24 (dd, J = 7.2, 2.1 Hz, 1H), 8.15 (d, J = 3.5 Hz, 1H), 7.39-7.36 |
| | | (m, 2H), 7.16-7.11 (m, 1H), 7.08 (s, 0.2H), 6.95 (s, 0.5H), 6.83 (s, 0.2H), |
| | | 5.86 (s, 2H), 3.53 (s, 2H), 2.31 (s, 6H); LRMS (ESI) m/z 430.34 (M⁺ + H). |
| 21 | 21 | 2-(6-((4-(3-(azetidin-1-ylmethyl)-4-fluorophenyl)-1H-1,2,3-triazol-1- |
| | | yl)methyl)pyridin-3-yl)-5-(difluoromethyl)-1,3,4-oxadiazole |
| | | ¹H NMR (400 MHz, CD₃OD) δ 9.28 (d, J = 1.6 Hz, 1H), 8.53 (dd, J = 8.4, |
| | | 2.4 Hz, 1H), 8.47 (d, J = 3.6 Hz, 1H), 8.11 (t, J = 7.8 Hz, 1H), 7.59 (d, J = 8.4 |
| | | Hz, 1H), 7.26 (t, J = 51.6 Hz, 1H), 7.25 (d, J = 8.0 Hz, 1H), 7.21 (d, J = 12.0 |
| | | Hz, 1H), 5.95 (s, 2H), 3.69 (s, 2H), 3.37~3.33 (m, 4H), 2.18~2.15 (m, |
| | | 2H); LRMS (ESI) m/z 443.0 (M⁺ + H). |
| 22 | 22 | 2-(difluoromethyl)-5-(6-((4-(4-fluoro-3-(pyrrolidin-1-ylmethyl)phenyl)-1H- |
| | | 1,2,3-triazol-1-yl)methyl)pyridin-3-yl)-1,3,4-oxadiazole |
| | | ¹H NMR (400 MHz, CD₃OD) δ 9.28 (d, J = 2.0 Hz, 1H), 8.53 (dd, J = 8.2, |
| | | 2.2 Hz, 1H), 8.47 (d, J = 3.6 Hz, 1H), 8.12 (t, J = 8.0 Hz, 1H), 7.59 (d, J = 8.4 |
| | | Hz, 1H), 7.32 (d, J = 1.2 Hz, 1H), 7.26 (t, J = 51.6 Hz, 1H), 7.29 (d, J = 11.2 |
| | | Hz, 1H), 5.95 (s, 2H), 3.72 (s, 2H), 2.30~2.25 (m, 4H), 1.88~1.84 (m, |
| | | 4H); LRMS (ESI) m/z 457.0 (M⁺ + H). |
| 23 | 23 | 1-(5-(1-((5-(5-(difluoromethyl)-1,3,4-oxadiazol-2-yl)pyridin-2-yl)methyl)- |
| | | 1H-1,2,3-triazol-4-yl)-2-fluorophenyl)-N,N-dimethylmethanamine |
| | | ¹H NMR (400 MHz, CD₃OD) δ 9.28 (d, J = 1.6 Hz, 1H), 8.53 (dd, J = 8.4, |
| | | 2.4 Hz, 1H), 8.48 (d, J = 3.6 Hz, 1H), 8.13 (t, J = 8.0 Hz, 1H), 7.28 (d, J = 7.6 |
| | | Hz, 1H), 7.25 (t, J = 51.6 Hz, 1H), 7.26 (d, J = 11.2 Hz, 1H), 5.95 (s, 2H), |
| | | 3.55 (s, 2H), 2.30 (s, 6H); LRMS (ESI) m/z 430.9 (M⁺ + H). |
| 24 | 24 | 2-(difluoromethyl)-5-(6-((4-(4-fluoro-3-(piperidin-1-ylmethyl)phenyl)-1H- |
| | | 1,2,3-triazol-1-yl)methyl)pyridin-3-yl)-1,3,4-oxadiazole |
| | | ¹H NMR (400 MHz, CD₃OD) δ 9.28 (d, J = 2.0 Hz, 1H), 8.53 (dd, J = 8.2, |
| | | 2.2 Hz, 1H), 8.47 (d, J = 3.6 Hz, 1H), 8.11 (t, J = 8.0 Hz, 1H), 7.59 (d, J = 8.4 |
| | | Hz, 1H), 7.29 (d, J = 8.0 Hz, 1H), 7.27 (d, J = 6.8 Hz, 1H), 7.26 (t, J = 51.6 |
| | | Hz, 1H), 5.95 (s, 2H), 3.57 (s, 2H), 2.48~2.42 (m, 4H), 1.67~1.61 (m, |
| | | 4H), 1.54~1.48 (m, 2H); LRMS (ESI) m/z 470.9 (M⁺ + H). |
| 25 | 25 | 2-(difluoromethyl)-5-(6-((4-(4-fluoro-3-((4-methylpiperidin-1- |
| | | yl)methyl)phenyl)-1H-1,2,3-triazol-1-yl)methyl)pyridin-3-yl)-1,3,4-oxadiazole |
| | | ¹H NMR (400 MHz, CD₃OD) δ 9.28 (d, J = 2.0 Hz, 1H), 8.53 (dd, J = 8.2, |
| | | 2.2 Hz, 1H), 8.47 (d, J = 3.6 Hz, 1H), 8.11 (t, J = 7.8 Hz, 1H), 7.59 (d, J = 8.4 |
| | | Hz, 1H), 7.29 (d, J = 3.2 Hz, 1H), 7.28 (d, J = 4.8 Hz, 1H), 7.26 (t, J = 51.6 |
| | | Hz, 1H), 5.95 (s, 2H), 3.58 (s, 2H), 2.91 (d, J = 11.6 Hz, 1H), 2.08 (t, |
| | | J = 10.6 Hz, 2H), 1.67 (d, J = 13.6 Hz, 2H), 1.43~1.39 (m, 1H), 1.33~1.23 |
| | | (m, 2H), 0.95 (d, J = 17.2 Hz, 3H); LRMS (ESI) m/z 484.9 (M⁺ + H). |
| 26 | 26 | 2-(6-((4-(3-(azetidin-1-ylmethyl)-2-fluorophenyl)-1H-1,2,3-triazol-1- |
| | | yl)methyl)pyridin-3-yl)-5-(difluoromethyl)-1,3,4-oxadiazole |
| | | ¹H NMR (400 MHz, CDCl₃) δ 9.35 (d, J = 1.8 Hz, 1H), 8.42 (dd, J = 8.2, 2.2 |
| | | Hz, 1H), 8.26-8.24 (m, 1H), 8.23-8.15 (m, 1H), 7.39-7.37 (m, 1H), 7.35-7.31 |
| | | (m, 1H), 7.26-7.22(m, 1H), 7.09 (s, 0.2H), 6.96 (s, 0.5H), 6.83 (s, |
| | | 0.2H), 5.85 (s, 2H), 3.70 (s, 2H), 3.34-3.30 (m, 4H), 2.17-2.10 (m, 2H); |
| | | LRMS (ESI) m/z 442.16 (M⁺ + H). |
| 27 | 27 | 2-(difluoromethyl)-5-(6-((4-(2-fluoro-3-(pyrrolidin-1-ylmethyl)phenyl)-1H- |
| | | 1,2,3-triazol-1-yl)methyl)pyridin-3-yl)-1,3,4-oxadiazole |
| | | ¹H NMR (400 MHz, CDCl₃) δ 9.35 (d, J = 2.0 Hz, 1H), 8.42-8.40 (m, 1H), |
| | | 8.27-8.25 (m, 1H), 8.23-8.16 (m, 1H), 7.43-7.38 (m, 2H), 7.27-7.23 |
| | | (m, 1H), 7.09 (s, 0.2H), 6.96 (s, 0.5H), 6.83 (s, 0.2H), 5.85 (s, 2H), 3.79 (s, |
| | | 2H), 2.64 (brs, 4H), 1.87-1.80 (m, 4H); LRMS (ESI) m/z 456.53 (M⁺ + H). |
| 28 | 28 | 2-(difluoromethyl)-5-(6-((4-(2-fluoro-3-(piperidin-1-ylmethyl)phenyl)-1H- |
| | | 1,2,3-triazol-1-yl)methyl)pyridin-3-yl)-1,3,4-oxadiazole |
| | | ¹H NMR (400 MHz, CDCl₃) δ 9.35 (d, J = 2.0 Hz, 1H), 8.42-8.40 (m, 1H), |
| | | 8.27-8.23 (m, 1H), 8.16 (d, J = 3.8 Hz, 1H), 7.42-7.39 (m, 2H), 7.27-7.23 |
| | | (m, 1H), 7.09 (s, 0.2H), 6.96 (s, 0.5H), 6.83 (s, 0.2H), 5.85 (s, 2H), 3.69 |
| | | (s, 2H), 2.52 (brs, 4H), 1.66-1.61 (m, 4H), 1.46-1.44 (m, 2H); LRMS |
| | | (ESI) m/z 470.05 (M⁺ + H). |
| 29 | 29 | 2-(difluoromethyl)-5-(6-((4-(2-fluoro-3-((4-methylpiperidin-1- |
| | | yl)methyl)phenyl)-1H-1,2,3-triazol-1-yl)methyl)pyridin-3-yl)-1,3,4-oxadiazole |
| | | ¹H NMR (400 MHz, CDCl₃) δ 9.35 (d, J = 2.1 Hz, 1H), 8.42-8.40 (m, 1H), |
| | | 8.26-8.24 (m, 1H), 8.22-8.15 (m, 1H), 7.40-7.38 (m, 2H), 7.26-7.22 |
| | | (m, 1H), 7.09 (s, 0.2H), 6.96 (s, 0.5H), 6.83 (s, 0.2H), 5.85 (s, 2H), 3.65 (s, |
| | | 2H), 2.94-2.92 (m, 2H), 2.07-2.04 (m, 2H), 1.64-1.61 (m, 2H), 1.38-1.25 |
| | | (m, 3H), 0.93 (d, J = 4.5 Hz, 3H); LRMS (ESI) m/z 484.42 (M⁺ + H). |
| 30 | 30 | 1-(3-(1-((5-(5-(difluoromethyl)-1,3,4-oxadiazol-2-yl)pyridin-2-yl)methyl)- |
| | | 1H-1,2,3-triazol-4-yl)-2-fluorophenyl)-N,N-dimethylmethanamine |
| | | ¹H NMR (400 MHz, CDCl₃) δ 9.35 (d, J = 2.0 Hz, 1H), 8.43-8.40 (m, 1H), |
| | | 8.30-8.26 (m, 1H), 8.17 (d, J = 3.8 Hz, 1H), 7.40-7.36 (m, 2H), 7.28-7.25 |
| | | (m, 1H), 7.09 (s, 0.2H), 6.96 (s, 0.5H), 6.83 (s, 0.2H), 5.85 (s, 2H), 3.62 |
| | | (s, 2H), 2.31 (s, 6H); LRMS (ESI) m/z 430.34 (M⁺ + H). |
| 31 | 31 | 1-(3-chloro-5-(1-((5-(5-(difluoromethyl)-1,3,4-oxadiazol-2-yl)pyridin-2- |
| | | yl)methyl)-1H-1,2,3-triazol-4-yl)phenyl)-N,N-dimethylmethanamine |
| | | ¹H NMR (400 MHz, CD₃OD) δ 9.28 (d, J = 2.1 Hz, 1H), 8.56 (s, 1H), 8.53 |
| | | (dd, J = 8.2, 2.2 Hz, 1H), 7.84 (t, J = 1.7 Hz, 1H), 7.76 (s, 1H), 7.61 (d, J = 8.2 |
| | | Hz, 1H), 7.38 (s, 1H), 7.26 (t, J = 51.5 Hz, 1H), 5.93 (s, 2H), 3.54 (s, 2H), |
| | | 2.29 (s, 6H); LRMS (ES) m/z 446.4 (M⁺ + 1). |
| 32 | 32 | 2-(6-((4-(3-chloro-5-(pyrrolidin-1-ylmethyl)phenyl)-1H-1,2,3-triazol-1- |
| | | yl)methyl)pyridin-3-yl)-5-(difluoromethyl)-1,3,4-oxadiazole |
| | | ¹H NMR (400 MHz, CD₃OD) δ 9.28 (d, J = 2.1 Hz, 1H), 8.56 (s, 1H), 8.53 |
| | | (dd, J = 8.2, 2.2 Hz, 1H), 7.83 (d, J = 1.6 Hz, 1H), 7.79 (s, 1H), 7.61 (d, J = 8.2 |
| | | Hz, 1H), 7.41 (s, 1H), 7.26 (t, J = 51.6 Hz, 1H), 5.93 (s, 2H), 3.72 (s, 2H), |
| | | 2.62 (s, 4H), 1.87~1.84 (m, 4H); LRMS (ES) m/z 472.4 (M⁺ + 1). |
| 33 | 33 | 2-(6-((4-(3-chloro-5-((4-methylpiperidin-1-yl)methyl)phenyl)-1H-1,2,3- |
| | | triazol-1-yl)methyl)pyridin-3-yl)-5-(difluoromethyl)-1,3,4-oxadiazole |
| | | ¹H NMR (400 MHz, CD₃OD) δ 9.27 (d, J = 2.0 Hz, 1H), 8.56 (s, 1H), 8.53 |
| | | (dd, J = 8.2, 2.2 Hz, 1H), 7.82~7.76 (m, 2H), 7.61 (d, J = 8.2 Hz, 1H), 7.39 |
| | | (s, 1H), 7.26 (t, J = 51.7 Hz, 1H), 5.93 (s, 2H), 3.58 (s, 2H), 2.91 (d, J = 11.6 |
| | | Hz, 2H), 2.09 (t, J = 10.8 Hz, 2H), 1.67 (d, J = 11.2 Hz, 2H), 1.44~1.38 (m, |
| | | 1H), 1.33~1.23 (m, 2H), 0.95 (d, J = 6.4 Hz, 3H); LRMS (ES) m/z 500.4 |
| | | (M⁺ + 1). |
| 34 | 34 | 1-(2-chloro-3-(1-((5-(5-(difluoromethyl)-1,3,4-oxadiazol-2-yl)pyridin-2- |
| | | yl)methyl)-1H-1,2,3-triazol-4-yl)phenyl)-N,N-dimethylmethanamine |
| | | ¹H NMR (400 MHz, CD₃OD) δ 9.28 (d, J = 2.0 Hz, 1H), 8.68 (s, 1H), 8.53 |
| | | (dd, J = 8.2, 2.2 Hz, 1H), 7.95 (dd, J = 7.7, 1.7 Hz, 1H), 7.60 (d, J = 8.3 Hz, |
| | | 1H), 7.53~7.51 (m, 1H), 7.44 (t, J = 7.7 Hz, 1H), 7.26 (t, J = 51.6 Hz, 1H), |
| | | 5.97 (s, 2H), 3.71 (s, 2H), 2.34 (s, 6H); LRMS (ES) m/z 446.4 (M⁺ + 1). |
| 35 | 35 | 2-(6-((4-(3-(azetidin-1-ylmethyl)-2-chlorophenyl)-1H-1,2,3-triazol-1- |
| | | yl)methyl)pyridin-3-yl)-5-(difluoromethyl)-1,3,4-oxadiazole |
| | | ¹H NMR (400 MHz, CD₃OD) δ 9.28 (d, J = 2.0 Hz, 1H), 8.67 (s, 1H), 8.53 |
| | | (dd, J = 8.2, 2.2 Hz, 1H), 7.94~7.92 (m, 1H), 7.60 (d, J = 8.2 Hz, 1H), |
| | | 7.47~7.41 (m, 2H), 7.26 (t, J = 51.6 Hz, 1H), 5.96 (s, 2H), 3.86 (s, 2H), |
| | | 3.42 (t, J = 7.2 Hz, 4H), 2.21~2.14 (m, 2H); LRMS (ES) m/z 458.4 (M⁺ + 1). |
| 36 | 36 | 2-(6-((4-(2-chloro-3-(pyrrolidin-1-ylmethyl)phenyl)-1H-1,2,3-triazol-1- |
| | | yl)methyl)pyridin-3-yl)-5-(difluoromethyl)-1,3,4-oxadiazole |
| | | ¹H NMR (400 MHz, CD₃OD) δ 9.28 (d, J = 2.0 Hz, 1H), 8.67 (s, 1H), 8.53 |
| | | (dd, J = 8.2, 2.3 Hz, 1H), 7.93 (dd, J = 7.8, 1.6 Hz, 1H), 7.61~7.55 (m, 2H), |
| | | 7.44 (t, J = 7.7 Hz, 1H), 7.26 (t, J = 51.6 Hz, 1H), 5.97 (s, 2H), 3.92 (s, 2H), |
| | | 2.70~2.68 (m, 4H), 1.88~1.84 (m, 4H); LRMS (ES) m/z 472.4 (M⁺ + 1). |
| 37 | 37 | 2-(6-((4-(2-chloro-3-((4-methylpiperidin-1-yl)methyl)phenyl)-1H-1,2,3- |
| | | triazol-1-yl)methyl)pyridin-3-yl)-5-(difluoromethyl)-1,3,4-oxadiazole |
| | | ¹H NMR (400 MHz, CD₃OD) δ 9.28 (d, J = 2.0 Hz, 1H), 8.66 (s, 1H), 8.53 |
| | | (dd, J = 8.2, 2.2 Hz, 1H), 7.91 (dd, J = 7.8, 1.5 Hz, 1H), 7.60~7.55 (m, 2H), |
| | | 7.43 (t, J = 7.7 Hz, 1H), 7.26 (t, J = 51.6 Hz, 1H), 5.96 (s, 2H), 3.73 (s, 2H), |
| | | 2.96 (d, J = 11.7 Hz, 2H), 2.19~2.13 (m, 2H), 1.66 (d, J = 13.3 Hz, 2H), |
| | | 1.45~1.39 (m, 1H), 1.34~1.24 (m, 2H), 0.96 (d, J = 6.4 Hz, 3H); LRMS |
| | | (ES) m/z 500.4 (M⁺ + 1). |
| 38 | 38 | 2-(6-((4-(3-(azetidin-1-ylmethyl)-5-fluorophenyl)-1H-1,2,3-triazol-1- |
| | | yl)methyl)pyridin-3-yl)-5-(difluoromethyl)-1,3,4-oxadiazole |
| | | ¹H NMR (400 MHz, CD₃OD) δ 9.28 (d, J = 2.0 Hz, 1H), 8.55 (s, 1H), 8.53 |
| | | (dd, J = 8.4, 2.4 Hz, 1H), 7.68~7.60 (m, 3H), 7.44 (t, J = 7.8 Hz, 1H), 7.26 |
| | | (t, J = 51.6 Hz, 1H), 5.93 (s, 2H), 3.72 (s, 2H), 3.39 (t, J = 7.2 Hz, 4H), |
| | | 2.18~2.11 (m, 2H); LRMS (ES) m/z 442.3 (M⁺ + 1). |
| 39 | 39 | 2-(difluoromethyl)-5-(6-((4-(3-fluoro-5-(pyrrolidin-1-ylmethyl)phenyl)-1H- |
| | | 1,2,3-triazol-1-yl)methyl)pyridin-3-yl)-1,3,4-oxadiazole |
| | | ¹H NMR (400 MHz, CD₃OD) δ 9.28 (d, J = 1.6 Hz, 1H), 8.56 (s, 1H), 8.53 |
| | | (dd, J = 8.2, 2.2 Hz, 1H), 7.68~7.60 (m, 3H), 7.52 (t, J = 7.8 Hz, 1H), 7.26 |
| | | (t, J = 51.6 Hz, 1H), 5.93 (s, 2H), 3.77 (s, 2H), 2.68~2.61 (m, 4H), 1.88~1.80 |
| | | (m, 4H); LRMS (ES) m/z 456.3 (M⁺ + 1). |
| 40 | 40 | 2-(difluoromethyl)-5-(6-((4-(2-(piperidin-1-ylmethyl)thiazol-4-yl)-1H- |
| | | 1,2,3-triazol-1-yl)methyl)pyridin-3-yl)-1,3,4-oxadiazole |
| | | ¹H NMR (400 MHz, CD₃OD) δ 9.28 (d, J = 2.4 Hz, 1H), 8.53 (dd, J = 8.0, |
| | | 2.0 Hz, 1H), 8.43 (s, 1H), 7.60 (d, J = 8.0 Hz, 1H), 7.26 (t, J = 51.6 Hz, 1H), |
| | | 5.94 (s, 2H), 3.87 (s, 2H), 2.61~2.54 (m, 4H), 1.69~1.63 (m, 4H), 1.54~1.51 |
| | | (m, 2H); LRMS (ES) m/z 459.4 (M⁺ + 1). |
| 41 | 41 | 1-(2-chloro-4-(1-((5-(5-(difluoromethyl)-1,3,4-oxadiazol-2-yl)pyridin-2- |
| | | yl)methyl)-1H-1,2,3-triazol-4-yl)phenyl)-N,N-dimethylmethanamine |
| | | ¹H NMR (400 MHz, CD₃OD) δ 9.28 (d, J = 2.0 Hz, 1H), 8.57 (s, 1H), 8.53 |
| | | (dd, J = 8.2, 2.2 Hz, 1H), 7.95 (d, J = 1.6 Hz, 1H), 7.80 (dd, J = 8.0, 1.6 Hz, |
| | | 1H), 7.61 (d, J = 8.3 Hz, 1H), 7.55 (d, J = 8.0 Hz, 1H), 7.26 (t, J = 51.6 Hz, |
| | | 1H), 5.93 (s, 2H), 3.66 (s, 2H), 2.33 (s, 6H); LRMS (ES) m/z 446.3 (M⁺ + 1). |
| 42 | 42 | 2-(6-((4-(4-(azetidin-1-ylmethyl)-3-chlorophenyl)-1H-1,2,3-triazol-1- |
| | | yl)methyl)pyridin-3-yl)-5-(difluoromethyl)-1,3,4-oxadiazole |
| | | ¹H NMR (400 MHz, CD₃OD) δ 9.28 (d, J = 1.8 Hz, 1H), 8.55 (s, 1H), 8.53 |
| | | (dd, J = 8.2, 2.1 Hz, 1H), 7.94 (d, J = 1.4 Hz, 1H), 7.80~7.78 (m, 1H), 7.60 |
| | | (d, J = 8.2 Hz, 1H), 7.48 (d, J = 8.0 Hz, 1H), 7.26 (t, J = 51.5 Hz, 1H), 5.92 |
| | | (s, 2H), 3.80 (s, 2H), 3.41 (t, J = 7.2 Hz, 4H), 2.20~2.13 (m, 2H); LRMS |
| | | (ES) m/z 458.3 (M⁺ + 1). |

### Protocol for Measuring and Analyzing Activity of Compound of Present Invention

### Experimental Example 1. Search for HDAC Enzyme Activity Inhibition (In Vitro)

An experiment was conducted to identify the selectivity of the 1,3,4-oxadiazole triazole compound of the present invention to HDAC6 through an experiment on HDAC1 and HDAC6 enzyme activity inhibition.

The HDAC enzyme activity was measured with HDAC Fluorimetric Drug Discovery Kit (BML-AK511, 516) of Enzo Life Science, Inc. For the test on the HDAC1 enzyme activity, human recombinant HDAC1 (BML-SE456) was used as an enzyme source and Fluor de Lys®-“SIRT1 (BNL-KI177)” was used as a substrate. A 5-fold dilution of the compound was divided into a 96-well plate, after which 0.3 μg of the enzyme and 10 μM of the substrate were inserted into each well and subjected to reaction at 30° C. for 60 minutes, such that Fluor de Lys® Developer II (BML-KI176) was inserted thereinto and subjected to reaction for 30 minutes and finished. After that, a fluorescence value (Ex 360, Em 460) was measured with a multi-plate reader (Flexstation 3, Molecular Device). An experiment on HDAC6 enzyme was conducted in accordance with the same protocol as in the HDAC1 enzyme activity test method by using human recombinant HDAC6 (382180) of Calbiochem Inc. For final result values, each IC₅₀ value was calculated with GraphPad Prism 4.0 program.

TABLE 6

| | | | HDAC6 | | | | HDAC6 |
|---|---|---|---|---|---|---|---|
| Compound | HDAC1 | HDAC6 | selectivity | Compound | HDAC1 | HDAC6 | selectivity |
| No. | (uM) | (uM) | (fold) | No. | (uM) | (uM) | (fold) |
| 1 | >50 | 23.5 | 2127 | 2 | >50 | 22.2 | 2252 |
| 3 | >50 | 29.2 | 1712 | 4 | >50 | 35.0 | 1428 |
| 5 | >50 | 32.3 | 1547 | 6 | >50 | 28.9 | 1730 |
| 7 | >50 | 39.9 | 1253 | 8 | >50 | 47.7 | 1048 |
| 9 | >50 | 32.7 | 1529 | 10 | >50 | 16.8 | 2976 |
| 11 | >50 | 31.7 | 1577 | 12 | >50 | 26.3 | 1901 |
| 13 | >50 | 26.4 | 1893 | 14 | >50 | 27.6 | 1811 |
| 15 | >50 | 24.4 | 2049 | 16 | >50 | 40.4 | 1237 |
| 17 | >50 | 34.1 | 1466 | 18 | >50 | 27.1 | 1845 |
| 19 | >50 | 33.5 | 1492 | 20 | >50 | 27.8 | 1798 |
| 21 | >50 | 13.4 | 3731 | 22 | >50 | 14.7 | 3401 |
| 23 | >50 | 18.5 | 2702 | 24 | >50 | 19.4 | 2577 |
| 25 | >50 | 17.3 | 2890 | 26 | >50 | 39.9 | 1253 |
| 27 | >50 | 34.2 | 1461 | 28 | >50 | 34.7 | 1440 |
| 29 | >50 | 42.9 | 1165 | 30 | >50 | 40.6 | 1231 |
| 31 | >50 | 35.1 | 1424 | 32 | >50 | 25.7 | 1945 |
| 33 | >50 | 37.3 | 1340 | 34 | >50 | 30.6 | 1633 |
| 35 | >50 | 43.6 | 1146 | 36 | >50 | 27.9 | 1792 |
| 37 | >50 | 43.1 | 1160 | 38 | >50 | 18.0 | 2777 |
| 39 | >50 | 28.2 | 1773 | 40 | >50 | 47.6 | 1050 |
| 41 | >50 | 24.0 | 2083 | 42 | >50 | 22.9 | 2183 |

As described in above table 6, it was confirmed from the results of testing the activity inhibition to HDAC1 and HDAC6 that the 1,3,4-oxadiazole triazole compound of the present invention, stereoisomers thereof or pharmaceutically acceptable salts thereof show about 1048 to about 3731 times more excellent selective HDAC6 inhibitory activity.

### Experimental Example 2. Analysis of Effect of HDAC6-Specific Inhibitor on Axonal Transport of Mitochondria (In Vitro)

By analyzing an effect of HDAC6-specific inhibitor on axonal transport of mitochondria, an experiment was performed to identify if the 1,3,4-oxadiazole triazole compound of the present invention selectively inhibits an HDAC6 activity and thus increases acetylation of tubulin, a key substrate of HDAC6 so as to show an effect of improving a transport velocity of mitochondria, which had been decreased by amyloid-beta treatment within a neuronal axon.

On the 17^{th} to 18^{th} days (E17-18) of insemination, the hippocampal neurons from a Sprague-Dawley (SD) rat fetus were cultured for seven days in a culture container for imaging, which had been coated with extracellular matrix, and were treated with amyloid-beta protein fragments at a concentration of 1M. In 24 hours later, the neurons were treated with the compound on the 8^{th} day of in vitro culture. In three hours later, the resulting neurons were treated with MitoTracker Red CMXRos (Life Technologies, NY, USA) for last five minutes to stain mitochondria. An image on the axonal transport of stained neuron mitochondria was taken with a confocal microscope (Leica SP8; Leica microsystems, UK) at an interval of one second for one minute to measure a transport velocity of each mitochondria per second with an IMARIS analysis program (BITPLANE, Zurich, Switzerland).

In result, after setting a section, in which the group treated with amyloid-beta had shown a significant decrease in the transport velocity of mitochondria compared to a vehicle, it was confirmed for the 1,3,4-oxadiazole triazole compound of the present invention, stereoisomers thereof or pharmaceutically acceptable salts thereof that the compound shows velocity distribution represented by *, 0% to 50%; **, 50% to 100%; ***, >100% after normalization with 100% of the vehicle and 0% of the amyloid beta treatment group.

TABLE 7

| | Velocity |
|---|---|
| | distribution |
| Classification | (%) |
| Vehicle | 100% |
| Amyloid beta | 0% |
| Compound 1 | *** |
| Compound 4 | * |
| Compound 11 | ** |
| Compound 18 | *** |
| Compound 21 | ** |
| Compound 22 | *** |
| Compound 23 | * |
| Compound 24 | ** |

1. A 1,3,4-oxadiazole triazole compound represented by formula I below, stereoisomers thereof, or pharmaceutically acceptable salts thereof: 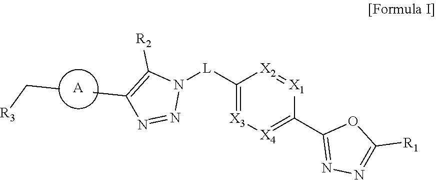
wherein,
X₁, X₂, X₃ and X₄ are each independently CH or N, in which at least one of X₁ to X₄ is N;
R₁ is CF₂H;
L is C1-C2 alkylene;
R₂ is H or C1-C5 alkyl;
A is C6-C12 aryl or 5 to 6 membered heteroaryl, in which at least one H of C6-C12 aryl is substituted with halogen;
R₃ is —NR₄R₅ or 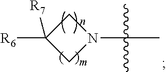
R₄ and R₅ are each independently H or C1-C6 alkyl;
R₆ and R₇ are each independently H, halogen, C1-C6 alkyl or C1-C6 haloalkyl; and
n and m are each independently 1 or 2.

2. The 1,3,4-oxadiazole triazole compound, stereoisomers thereof, or pharmaceutically acceptable salts thereof according to claim 1, wherein:
X₁, X₃ and X₄ of formula I are each CH, and X₂ is N;
L is C1 alkylene; and
R₁, R₂, A and R₃ are as defined in claim 1, respectively.

3. The 1,3,4-oxadiazole triazole compound, stereoisomers thereof, or pharmaceutically acceptable salts thereof according to claim 1, wherein:
X₁ to X₄, R₁, L and R₂ of formula I are as defined in claim 1;
A is C6 aryl, in which at least one H of C6 aryl is substituted with halogen;
R₃ is —NR₄R₅ or 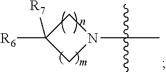
R₄ and R₅ are each independently C1-C6 alkyl;
R₆ and R₇ are each independently H or C1-C6 alkyl; and
n and m are each independently 1 or 2.

4. The 1,3,4-oxadiazole triazole compound, stereoisomers thereof, or pharmaceutically acceptable salts thereof according to claim 1, wherein:
X₁ to X₄, R₁, L and R₂ of formula I are as defined in claim 1;
A is 6-membered heteroaryl;
R₃ is 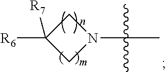
R₆ and R₇ are each independently H or C1-C6 alkyl; and
n and m are each independently 1 or 2.

5. The 1,3,4-oxadiazole triazole compound, stereoisomers thereof, or pharmaceutically acceptable salts thereof according to claim 1, wherein:
X₁ to X₄, R₁, L and R₂ of above formula I are as defined in claim 1;
A is 5-membered heteroaryl; 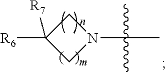
R₃ is —NR₄R₅ or
R₄ and R₅ are each independently C1-C6 alkyl;
R₆ and R₇ are each independently H, halogen, C1-C6 alkyl or C1-C6 haloalkyl; and
n and m are each independently 1 or 2.

6. A 1,3,4-oxadiazole triazole compound selected from the group consisting of compounds shown in the following table, stereoisomers thereof, or pharmaceutically acceptable salts thereof:

| Com- | |
|---|---|
| pound | |
| No. | Structure |
| 1 | 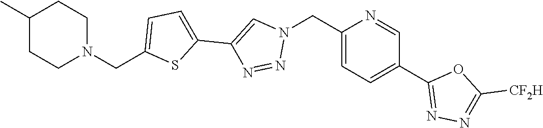 [SMILES: CC1CCN(Cc2ccc(-c3cn(Cc4ccc(-c5nnc(C(F)F)o5)cn4)nn3)s2)CC1] |
| 2 | 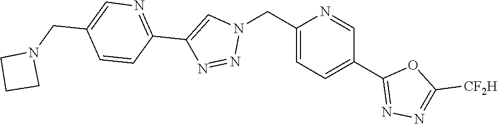 [SMILES: FC(F)c1nnc(-c2ccc(Cn3cc(-c4ccc(CN5CCC5)cn4)nn3)nc2)o1] |
| 3 | 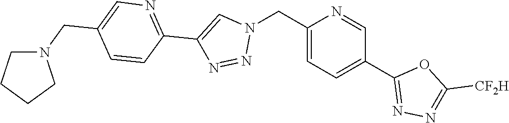 [SMILES: FC(F)c1nnc(-c2ccc(Cn3cc(-c4ccc(CN5CCCC5)cn4)nn3)nc2)o1] |
| 4 | 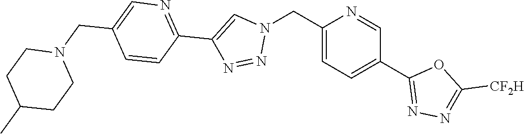 [SMILES: CC1CCN(Cc2ccc(-c3cn(Cc4ccc(-c5nnc(C(F)F)o5)cn4)nn3)nc2)CC1] |
| 5 | 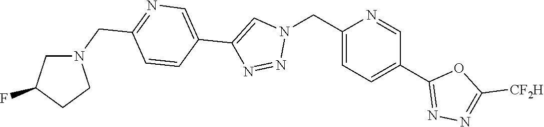 [SMILES: FC(F)c1nnc(-c2ccc(Cn3cc(-c4ccc(CN5CC[C@@H](F)C5)nc4)nn3)nc2)o1] |
| 6 | 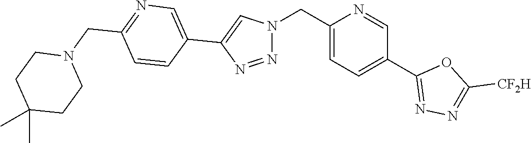 [SMILES: CC1(C)CCN(Cc2ccc(-c3cn(Cc4ccc(-c5nnc(C(F)F)o5)cn4)nn3)cn2)CC1] |
| 7 | 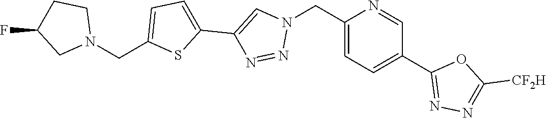 [SMILES: FC(F)c1nnc(-c2ccc(Cn3cc(-c4ccc(CN5CC[C@H](F)C5)s4)nn3)nc2)o1] |
| 8 | 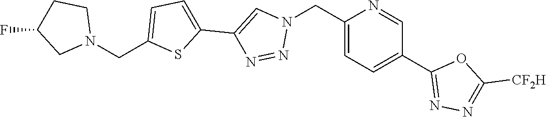 [SMILES: FC(F)c1nnc(-c2ccc(Cn3cc(-c4ccc(CN5CC[C@@H](F)C5)s4)nn3)nc2)o1] |
| 9 | 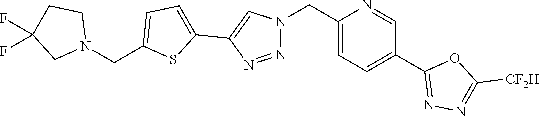 [SMILES: FC(F)c1nnc(-c2ccc(Cn3cc(-c4ccc(CN5CCC(F)(F)C5)s4)nn3)nc2)o1] |
| 10 | 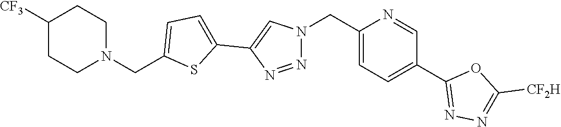 [SMILES: FC(F)c1nnc(-c2ccc(Cn3cc(-c4ccc(CN5CCC(C(F)(F)F)CC5)s4)nn3)nc2)o1] |
| 11 | 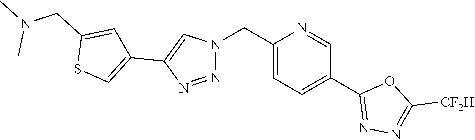 [SMILES: CN(C)Cc1cc(-c2cn(Cc3ccc(-c4nnc(C(F)F)o4)cn3)nn2)cs1] |
| 12 | 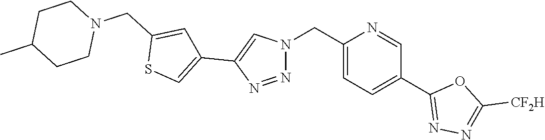 [SMILES: CC1CCN(Cc2cc(-c3cn(Cc4ccc(-c5nnc(C(F)F)o5)cn4)nn3)cs2)CC1] |
| 13 | 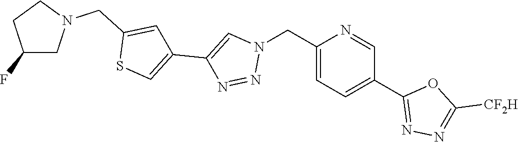 [SMILES: FC(F)c1nnc(-c2ccc(Cn3cc(-c4csc(CN5CC[C@H](F)C5)c4)nn3)nc2)o1] |
| 14 | 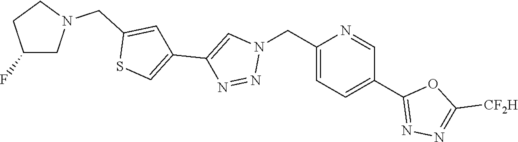 [SMILES: FC(F)c1nnc(-c2ccc(Cn3cc(-c4csc(CN5CC[C@@H](F)C5)c4)nn3)nc2)o1] |
| 15 | 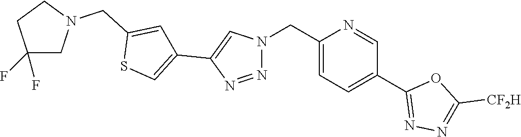 [SMILES: FC(F)c1nnc(-c2ccc(Cn3cc(-c4csc(CN5CCC(F)(F)C5)c4)nn3)nc2)o1] |
| 16 | 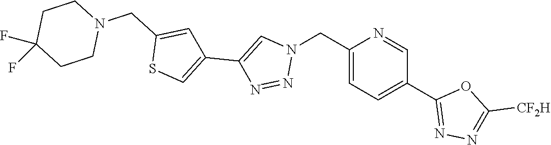 [SMILES: FC(F)c1nnc(-c2ccc(Cn3cc(-c4csc(CN5CCC(F)(F)CC5)c4)nn3)nc2)o1] |
| 17 | 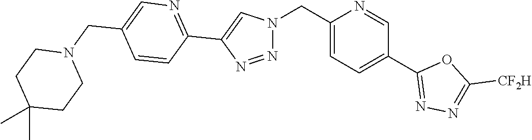 [SMILES: CC1(C)CCN(Cc2ccc(-c3cn(Cc4ccc(-c5nnc(C(F)F)o5)cn4)nn3)nc2)CC1] |
| 18 | 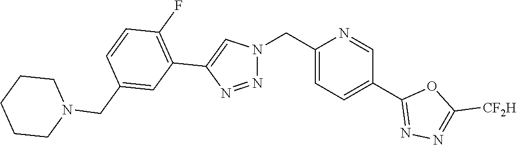 [SMILES: Fc1ccc(CN2CCCCC2)cc1-c1cn(Cc2ccc(-c3nnc(C(F)F)o3)cn2)nn1] |
| 19 | 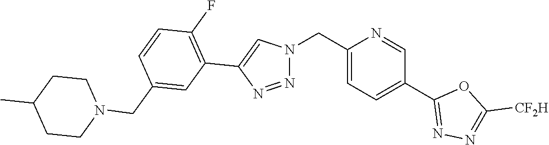 [SMILES: CC1CCN(Cc2ccc(F)c(-c3cn(Cc4ccc(-c5nnc(C(F)F)o5)cn4)nn3)c2)CC1] |
| 20 | 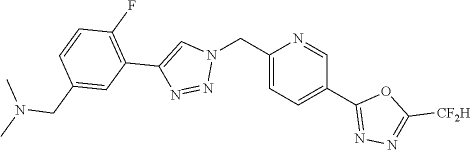 [SMILES: CN(C)Cc1ccc(F)c(-c2cn(Cc3ccc(-c4nnc(C(F)F)o4)cn3)nn2)c1] |
| 21 | 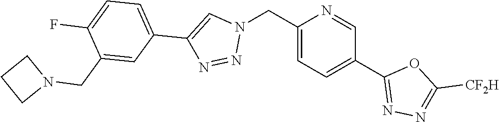 [SMILES: Fc1ccc(-c2cn(Cc3ccc(-c4nnc(C(F)F)o4)cn3)nn2)cc1CN1CCC1] |
| 22 | 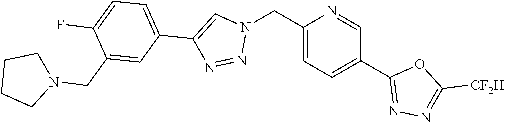 [SMILES: Fc1ccc(-c2cn(Cc3ccc(-c4nnc(C(F)F)o4)cn3)nn2)cc1CN1CCCC1] |
| 23 | 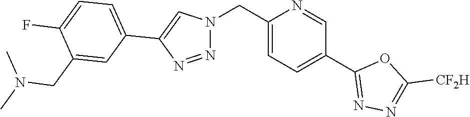 [SMILES: CN(C)Cc1cc(-c2cn(Cc3ccc(-c4nnc(C(F)F)o4)cn3)nn2)ccc1F] |
| 24 | 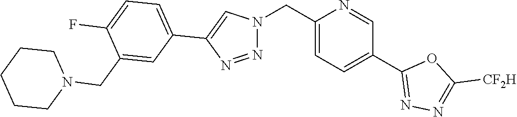 [SMILES: Fc1ccc(-c2cn(Cc3ccc(-c4nnc(C(F)F)o4)cn3)nn2)cc1CN1CCCCC1] |
| 25 | 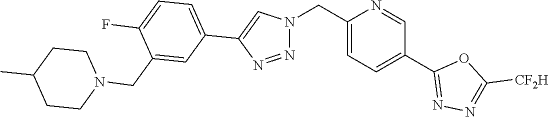 [SMILES: CC1CCN(Cc2cc(-c3cn(Cc4ccc(-c5nnc(C(F)F)o5)cn4)nn3)ccc2F)CC1] |
| 26 | 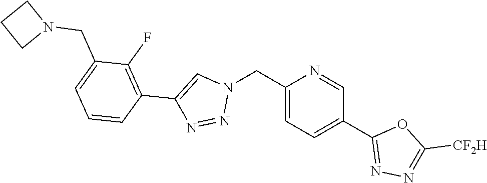 [SMILES: Fc1c(CN2CCC2)cccc1-c1cn(Cc2ccc(-c3nnc(C(F)F)o3)cn2)nn1] |
| 27 | 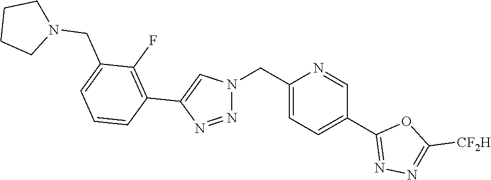 [SMILES: Fc1c(CN2CCCC2)cccc1-c1cn(Cc2ccc(-c3nnc(C(F)F)o3)cn2)nn1] |
| 28 | 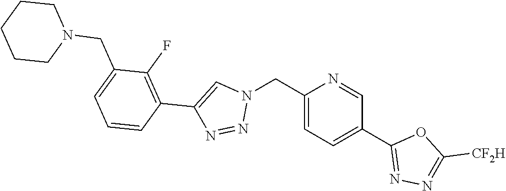 [SMILES: Fc1c(CN2CCCCC2)cccc1-c1cn(Cc2ccc(-c3nnc(C(F)F)o3)cn2)nn1] |
| 29 | 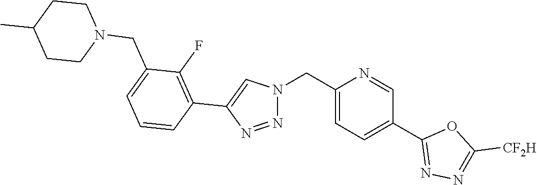 [SMILES: CC1CCN(Cc2cccc(-c3cn(Cc4ccc(-c5nnc(C(F)F)o5)cn4)nn3)c2F)CC1] |
| 30 | 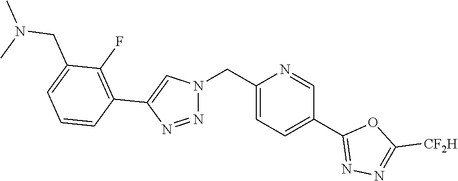 [SMILES: CN(C)Cc1cccc(-c2cn(Cc3ccc(-c4nnc(C(F)F)o4)cn3)nn2)c1F] |
| 31 | 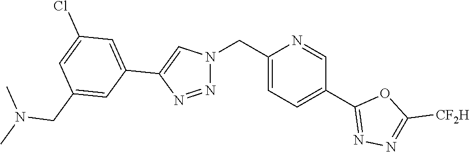 [SMILES: CN(C)Cc1cc(Cl)cc(-c2cn(Cc3ccc(-c4nnc(C(F)F)o4)cn3)nn2)c1] |
| 32 | 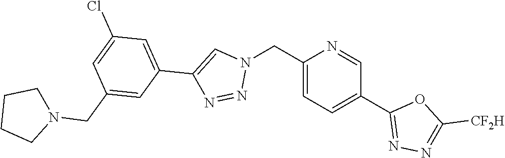 [SMILES: FC(F)c1nnc(-c2ccc(Cn3cc(-c4cc(Cl)cc(CN5CCCC5)c4)nn3)nc2)o1] |
| 33 | 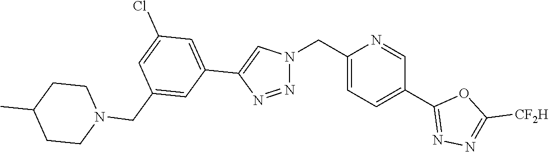 [SMILES: CC1CCN(Cc2cc(Cl)cc(-c3cn(Cc4ccc(-c5nnc(C(F)F)o5)cn4)nn3)c2)CC1] |
| 34 | 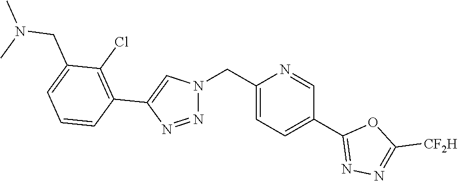 [SMILES: CN(C)Cc1cccc(-c2cn(Cc3ccc(-c4nnc(C(F)F)o4)cn3)nn2)c1Cl] |
| 35 | 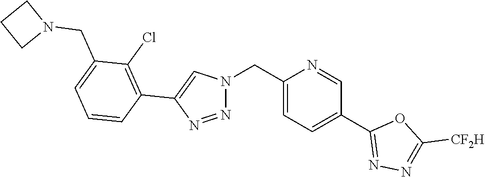 [SMILES: FC(F)c1nnc(-c2ccc(Cn3cc(-c4cccc(CN5CCC5)c4Cl)nn3)nc2)o1] |
| 36 | 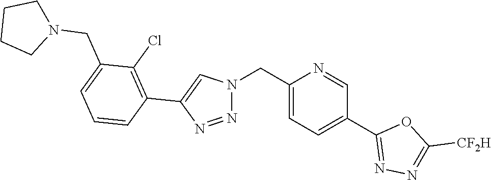 [SMILES: FC(F)c1nnc(-c2ccc(Cn3cc(-c4cccc(CN5CCCC5)c4Cl)nn3)nc2)o1] |
| 37 | 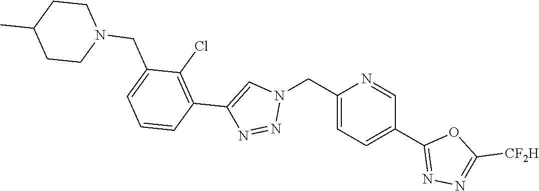 [SMILES: CC1CCN(Cc2cccc(-c3cn(Cc4ccc(-c5nnc(C(F)F)o5)cn4)nn3)c2Cl)CC1] |
| 38 | 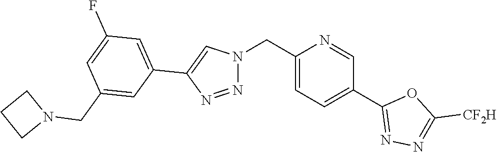 [SMILES: Fc1cc(CN2CCC2)cc(-c2cn(Cc3ccc(-c4nnc(C(F)F)o4)cn3)nn2)c1] |
| 39 | 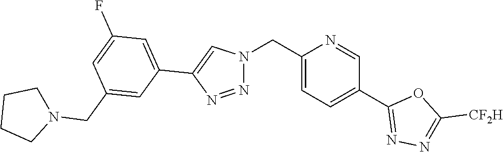 [SMILES: Fc1cc(CN2CCCC2)cc(-c2cn(Cc3ccc(-c4nnc(C(F)F)o4)cn3)nn2)c1] |
| 40 | 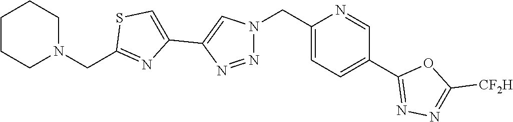 [SMILES: FC(F)c1nnc(-c2ccc(Cn3cc(-c4csc(CN5CCCCC5)n4)nn3)nc2)o1] |
| 41 | 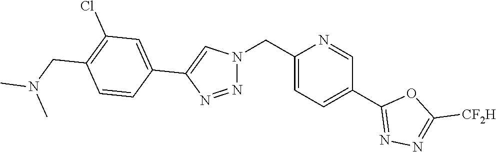 [SMILES: CN(C)Cc1ccc(-c2cn(Cc3ccc(-c4nnc(C(F)F)o4)cn3)nn2)cc1Cl] |
| 42 | 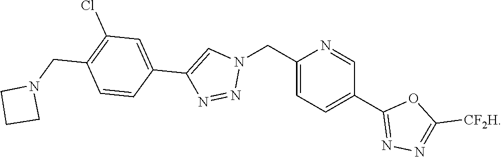 [SMILES: FC(F)c1nnc(-c2ccc(Cn3cc(-c4ccc(CN5CCC5)c(Cl)c4)nn3)nc2)o1] |

7. A pharmaceutical composition for preventing or treating a histone deacetylase-mediated disease, the composition comprising a 1,3,4-oxadiazole triazole compound, stereoisomer thereof or pharmaceutically acceptable salt thereof according to claim 1 and a pharmaceutically acceptable carrier.

8. A method for the prevention or treatment of a histone deacetylase-mediated disease in a subject in need thereof, comprising administering to the subject a therapeutically effective amount of a 1,3,4-oxadiazole triazole compound, stereoisomer thereof or pharmaceutically acceptable salt thereof according to claim 1, wherein the histone deacetylase-mediated disease is an infectious disease; a neoplasm, endocrinopathy, a nutritional or metabolic disease; a mental or behavioral disorder, a neurological disease, an eye or ocular adnexal disease; a respiratory disease; a digestive troubles, a skin or subcutaneous tissue disease; a musculoskeletal system or connective tissue disease; or a teratosis, deformity or chromosomal aberration.

9. The method according to claim 8, wherein:
the endocrinopathy, nutritional and metabolic disease is Wilson's disease, amyloidosis or diabetes;
the mental and behavioral disorder is depression or Rett syndrome;
the neurological disease is central nervous system atrophy, neurodegenerative disease, motor disorder, neuropathy, motor neuron disease or central nervous system demyelinating disease;
the eye and ocular adnexal disease is uveitis;
the skin and subcutaneous tissue disease is psoriasis;
the musculoskeletal system and connective tissue disease is rheumatoid arthritis, osteoarthritis or systemic lupus erythematosis;
the teratosis, deformities and chromosomal aberration is autosomal dominant polycystic kidney disease;
the infectious disease is prion disease;
the neoplasm is benign tumor or malignant tumor;
the respiratory disease is asthma; and
the digestive troubles are is alcoholic liver disease, inflammatory bowel disease, Crohn's disease or ulcerative bowel disease.

10-11. (canceled)

12. A method for preventing or treating a histone deacetylase-mediated disease in a subject in need thereof, the method comprising administering to the subject a therapeutically effective amount of the 1,3,4-oxadiazole triazole compound, stereoisomer thereof, or pharmaceutically acceptable salt thereof according to claim 2.

13. A method for preventing or treating a histone deacetylase-mediated disease in a subject in need thereof, the method comprising administering to the subject a therapeutically effective amount of the 1,3,4-oxadiazole triazole compound, stereoisomer thereof, or pharmaceutically acceptable salt thereof according to claim 2.

14. A method for preventing or treating a histone deacetylase-mediated disease in a subject in need thereof, the method comprising administering to the subject a therapeutically effective amount of the 1,3,4-oxadiazole triazole compound, stereoisomer thereof, or pharmaceutically acceptable salt thereof according to claim 3.

15. A method for preventing or treating a histone deacetylase-mediated disease in a subject in need thereof, the method comprising administering to the subject a therapeutically effective amount of the 1,3,4-oxadiazole triazole compound, stereoisomer thereof, or pharmaceutically acceptable salt thereof according to claim 4.

16. A method for preventing or treating a histone deacetylase-mediated disease in a subject in need thereof, the method comprising administering to the subject a therapeutically effective amount of the 1,3,4-oxadiazole triazole compound, stereoisomer thereof, or pharmaceutically acceptable salt thereof according to claim 5.

17. A method for preventing or treating a histone deacetylase-mediated disease in a subject in need thereof, the method comprising administering to the subject a therapeutically effective amount of the 1,3,4-oxadiazole triazole compound, stereoisomer thereof, or pharmaceutically acceptable salt thereof according to claim 6.

18. A pharmaceutical composition for preventing or treating histone deacetylase-mediated diseases, the composition comprising a 1,3,4-oxadiazole triazole compound, stereoisomer thereof or pharmaceutically acceptable salt thereof according to claim 2 and a pharmaceutically acceptable carrier.

19. A pharmaceutical composition for preventing or treating histone deacetylase-mediated diseases, the composition comprising a 1,3,4-oxadiazole triazole compound, stereoisomer thereof or pharmaceutically acceptable salt thereof according to claim 3 and a pharmaceutically acceptable carrier.

20. A pharmaceutical composition for preventing or treating histone deacetylase-mediated diseases, the composition comprising a 1,3,4-oxadiazole triazole compound, stereoisomer thereof or pharmaceutically acceptable salt thereof according to claim 4 and a pharmaceutically acceptable carrier.

21. A pharmaceutical composition for preventing or treating histone deacetylase-mediated diseases, the composition comprising a 1,3,4-oxadiazole triazole compound, stereoisomer thereof or pharmaceutically acceptable salt thereof according to claim 5 and a pharmaceutically acceptable carrier.

22. A pharmaceutical composition for preventing or treating histone deacetylase-mediated diseases, the composition comprising a 1,3,4-oxadiazole triazole compound, stereoisomer thereof or pharmaceutically acceptable salt thereof according to claim 6 and a pharmaceutically acceptable carrier.
